(19) United States
(12) Patent Application Publication
(10) Pub. No.: US 2014/0243415 A1
(43) Pub. Date: Aug. 28, 2014

(54) COMPOUNDS INHIBITING GALECTIN-1 EXPRESSION, CANCER CELL PROLIFERATION, INVASION, AND TUMORIGENESIS

(71) Applicants: Eric Raymond, Clichy (FR); Lucile Astorgues-Xerri, Clichy (FR); Sandrine Faivre, Clichy (FR); Esteban Cvitkovic, Clichy (FR)

(72) Inventors: Eric Raymond, Clichy (FR); Lucile Astorgues-Xerri, Clichy (FR); Sandrine Faivre, Clichy (FR); Esteban Cvitkovic, Clichy (FR)

(73) Assignee: ONCOETHIX SA, Lousanne (CH)

(21) Appl. No.: 14/008,339

(22) PCT Filed: Mar. 30, 2012
(86) PCT No.: PCT/EP12/55902

§ 371 Date: May 8, 2014

Related U.S. Application Data
(60) Provisional application No. 61/469,462, filed on Mar. 30, 2011.
(60) Provisional application No. 61/470,301, filed on Mar. 31, 2011.
(60) Provisional application No. 61/562,819, filed on Nov. 22, 2011.
(60) Provisional application No. 61/558,144, filed on Nov. 10, 2011.

(51) Int. Cl. A61K 31/165; A61K 45/06

(52) U.S. Cl. CPC A61K 31/165; A61K 45/06
USPC 514/622; 435/375

(57) ABSTRACT

Disclosed herein are compositions comprising a galectin-1-targeting compound in a therapeutically effective composition for treating cancer. In an aspect, a galectin-1-targeting compound is OTX-008. Also disclosed herein are methods of making and using such compositions.

### BACKGROUND

Galectins are a structurally related family of lectins defined by their affinity for β-galactoside glycans. Galectin-1 was first described as a determinant factor in cancer cell adhesion but was ultimately recognized as a multifunctional protein involved in various aspects of tumorigenesis, cell-extracellular matrix and cell-cell interactions, cell migration, angiogenesis, and immune surveillance escape (A. Danguy et al., Biochimica et Biophysica Acta (2002) 1572:285-293). Up-regulation of galectin-1 was associated with poor prognosis and acquisition of a metastatic phenotype in several tumor models. Consistently, exposure to exogenous galectin-1 was shown to promote metastasis by positively regulating cell migration and invasiveness. Galectin-1 expression has been examined in several malignant tumors. Correlations between galectin-1 expression, tumor invasiveness and lymph node metastasis were demonstrated in hepatocarcinoma, breast cancer, neuroblastoma, lung adenocarcinoma, and head and neck squamous cell carcinomas. Cancer cells that secrete galectin-1 may form extracellular lattices, which facilitate growth factor/receptor and cell/matrix interactions. Galectin-1 was shown to enhance VEGFR-dependent angiogenesis through interaction with neuropilin-1. The binding of galectin-1 to neuropilin-1 enhances VEGFR2 phosphorylation and stimulates the activation of the MAPK pathways. Moreover, galectin-1 is highly up-regulated in tumor-activated endothelial cells and is crucial for activated endothelial cells to adhere to, and to migrate on the extracellular matrix. More recently, reports accumulated regarding the interactions between intracytoplasmic galectin-1 and Ras-dependent signaling cascades. Galectin-1 was shown to bind oncogenic H-Ras, increased membrane associated Ras-GTP facilitating Raf activation, inhibited caveolin formation, and promoted dissociation of Ras anchorage from the plasma membrane toward the endoplasmic Golgi and reticulums. These processes were shown to be critical in the oncogenic role of Ras as well as in downstream activation of Raf-1 and ERK1/2. Based on those background data, galectin-1 has been considered as an attractive target for the treatment of cancer.

Anginex, a synthetic 33-amino acid peptide modeled to reproduce the β-sheet structure of platelet factor 4 and interleukin-8, was the first agent designed to inhibit galectin-1. So far, most data have focused on the antiangiogenic properties of anginex showing that the drug operates against tumor-activated endothelial cells via prevention of cell adhesion/migration on the extracellular matrix and inhibition of endothelial cell proliferation via anoikis and apoptosis. Anginex also shows antitumor activity in the syngeneic mouse B16 melanoma model and in human ovarian carcinoma MA148 xenografts. A non-peptidic topomimetic of anginex, OTX-008 (FIG. 1A), was shown to be a potent angiogenesis inhibitor in vitro, as determined by endothelial cell proliferation, migration, and chick embryo chorioallantoic membrane assays.

### SUMMARY

In an embodiment, a method of inhibiting a galectin-1-associated pathway in a mammalian cell comprises contacting a mammalian cell with an effective amount of a galectin-1-targeting compound. In an embodiment, the method is a method of treating cancer in a subject having cancer, comprising contacting one or more cancerous cells in the subject with a therapeutically effective amount of a galectin-1 targeting compound. In an embodiment, the method is a method of inhibiting at least one of growth and proliferation of a cancer cell and includes contacting a cancer cell with a therapeutically effective amount of a galectin-1 targeting compound.

In another embodiment, a method of inhibiting a galectin-1-associated pathway in a mammalian cell comprises contacting a mammalian cell with an effective amount of a galectin-1-targeting compound and inhibiting at least one of tumor growth and metastasis in a patient having a tumor, the method including contacting a tumor with a therapeutically effective amount of a galectin-1 targeting compound by inhibiting at least one of galectin-1 activity and expression, by contacting a cell with a therapeutically effective amount of a galectin-1 targeting compound.

In an embodiment, a method of inhibiting a galectin-1-associated pathway in a mammalian cell comprises contacting a mammalian cell with an effective amount of OTX-008. In an embodiment, a galectin-1 targeting compound is OTX-008.

In an embodiment, a method of inhibiting a galectin-1-associated pathway in a mammalian cell includes contacting a mammalian cell with an effective amount of OTX-008. In an embodiment, the method is a method of treating cancer in a subject having cancer, comprising contacting one or more cancerous cells in the subject with a therapeutically effective amount of OTX-008. In another embodiment, the method is a method of inhibiting at least one of growth and proliferation of a cancer cell comprising contacting a cancer cell with a therapeutically effective amount of OTX-008. In another embodiment, the methods is a method of inhibiting at least one of tumor growth and metastasis in a patient having a tumor, comprising contacting said tumor with a therapeutically effective amount of OTX-008. In yet another embodiment, the method is a method of inhibiting at least one of galectin-1 activity and expression, comprising contacting a cell with a therapeutically effective amount of OTX-008.

In an embodiment, a method of treating cancer in a subject having cancer comprises contacting one or more cancerous cells in the subject with a therapeutically effective amount of a galectin-1 targeting compound, wherein the treatment of the cancer includes at least one of preventing or slowing the growth of the cancer, preventing the spread of a tumor associated with the cancer, preventing the spread of one or more metastases associated with the cancer, reducing the size of a tumor associated with the cancer, and preventing the recurrence of cancer treated previously. In an embodiment, the therapeutically effective amount of OTX-008 comprises a pharmaceutical composition.

In an embodiment, a pharmaceutical composition encompassed herein is administered by at least one route selected from the group consisting of intravenously, subcutaneously, intradermally, parenterally, and intramuscularly. In an embodiment, the pharmaceutical composition is administered in combination with at least one other mode of therapy selected from the group consisting of radiotherapy, chemotherapy, and surgery.

In an embodiment, a method is provided for identifying a cancer cell susceptible to treatment with a galectin-1-targeting compound, comprising identifying in the cell at least one marker selected from the group consisting of higher than normal E-cadherin expression level, lower than normal vimentin expression level, lower than normal galectin-1 expression level, higher than normal keratin-8 expression level, and higher than normal keratin-18 expression level.

In an embodiment, a method and composition as described herein are provided for treatment of one or more of ovarian cancer, squamous cell carcinoma, a cancer of the digestive system, stomach cancer, liver cancer, colon cancer, a cancer of the thyroid, a cancer of the endometrium, adenocarcinoma of the endometrium, uterine cancer, uterine adenocarcinomac a uterine smooth muscle tumor, breast cancer, prostate cancer, bladder cancer, a head cancer, a neck cancer, a glioma, a kidney cancer, pancreatic cancer, pancreatic ductal adenocarcinoma, nonsmall-cell lung cancer, and melanoma.

In an embodiment, a method of inhibiting the galectin-1/semaphorin-3A system in a cancer cell comprises contacting the cell with an effective amount of a galectin-1-targeting compound. In another embodiment, a method of inhibiting a pathway in a cell comprises contacting the cell with an effective amount of a galectin-1-targeting compound, wherein the pathway is at least one of the members selected from the group consisting of an ERK1/2-phosphorylation pathway, an AKT-phosphorylation pathway, an S6-phosphorylation pathway, a MAPK pathway, and a p-wee1 pathway.

In an embodiment, in a method described herein using a galectin-1 targeting compound, the galectin-1-targeting compound is administered in a dose selected from the group consisting of about 1 microgram to about 1,000 micrograms, about 25 micrograms to about 500 micrograms, about 50 to about 250 micrograms, about 2 micrograms to about 100 micrograms, about 5 micrograms to about 75 micrograms, about 10 micrograms to about 50 micrograms, and about 20 micrograms to about 40 micrograms.

In an embodiment, a method of treating a galectin-1-overexpressing cancer in a patient having a galectin-1-overexpressing cancer comprises administering to the patient a therapeutically effective amount of a composition comprising at least one galectin-1-targeting compound.

### BRIEF DESCRIPTION OF THE DRAWINGS

FIG. 1. Expression of galectin-1 correlates with OTX-008 sensitivity in cancer cells. A—Chemical structure of OTX-008, a calix(4)arene protein surface topomimetic. B—Antiproliferative effects of OTX-008 was determined using MTT assay in one head and neck tumor cell, SQ20B, and two ovarian tumor cells, OVCAR3 and A2780-1A9, exposed to 4 drug concentrations. C—Correlation between OTX-008 IC50s and galectin-1 (LGALS1) relative mRNA expression.

FIG. 2. Epithelial-to-mesenchymal differentiation and OTX-008 sensitivity. A—Correlations between galectin-1 (LGALS1) and vimentin, E-cadherin (CDH1) and N-cadherin (CDH2) relative mRNA expression. B—Correlations between OTX-008 IC50s and vimentin, E-cadherin (CDH1) and N-cadherin (CDH2) relative mRNA expression. C—Antiproliferative effects of OTX-008 in epithelial COLO205-S and mesenchymal COLO205-R colon cancer cells. D—Antiproliferative effects of OTX-008 in SNAIL-inducible colon cancer cell lines. E—Antiproliferative effects of OTX-008 in shWISP breast cancer cell lines.

FIG. 3. Effects of OTX-008 on galectin-1 expression and localization. A—Western Blot of galectin-1 on SQ20B cells treated with 3 μM OTX-008 for 24 h, 48 h and 72 h. B and C—Immunofluorescence of galectin-1 on SQ20B cells treated with 3 μM OTX-008 for 24 and 48 h. B—OTX-008 treatment decreases galectin-1 immunostaining in SQ20B cells. C—Galectin-1 translocates into the nucleus after OTX-008 exposure. D—Western Blot of p-AKTser473, AKT, p-S6, p-ERK1/2, ERK and p-PKCα in SQ20B cells treated with 3 μM OTX-008 for 10 min, 1 h, 2 h, 5 h and 24 h.

FIG. 4. OTX-008-induced galectin-1 oxydation and degradation by the proteosome system A—Western Blot of galectin-1 on SQ20B cells treated 48 h with 10 μM N-acetylcysteine (NAC), 55 μM β-mercaptoethanol (βME), 1 mM Tempol and 10 μM co-enzyme Q10 (Q10) with or without 3 μM OTX-008. B—Western Blot of galectin-1 on SQ20B cells treated 48 h with 3 μM OTX-008, pretreated 2 h with proteasome inhibitor Bortezomib (10 nM) and then treated 48 h with 3 μM OTX-008 (BTZ—OTX-008) or treated 48 h with 10 nM Bortezomib+3 μM OTX-008 (BTZ+OTX-008).

FIG. 5. Modulation of the galectin-1/semaphorin-3A/neuropilin system in SQ20B cells. A—Effects of galectin-1 (Gall), semaphorin-3A (Sema3A), lactose, with or without four different concentrations of OTX-008 on SQ20B growth at day 6. Embedded figure: Proliferation assay of SQ20B and SQ-shGAL1. B—In vitro matrigel invasion assay of SQ20B exposed to 1 g/ml galectin-1, 1 μg/ml semaphorin-3A, 200 ng/ml lactose or 10 μM UO126 with or without 3 μM OTX-008. Embedded figure: In vitro matrigel invasion assay of SQ20B and SQ-shGAL1. Invasion was quantified after 48 h by counting the average number of invaded cells.

FIG. 6. Antitumor and antiangiogenic effects of OTX-008 at non-toxic doses in A2780-1A9 and SQ20B human tumor xenografts. A—The antitumor effects of OTX-008 on A2780-1A9 xenograft were compared to that of active doses of cisplatin and paclitaxel—solid line represents days of treatment. B—Immunohistochemistry of galectin-1, Ki67 (MIB-1) and CD31 on A2780-1A9 xenograft. C—Tumor growth and secondary tumor formation of SQ20B xenografts untreated and treated with 10 mg/kg/day OTX-008 and SQ-shGAL1 xenografts. Tumor volume was determined as 3.14(width2)/length—solid line represents days of treatment. D—Immunohistochemistry of galectin-1, MIB1, VEGFR2 and CD31 on SQ20B xenografts

FIG. 7. Modulation of the galectin-1/semaphorin-3A/neuropilin system in SQ20B cells. A—Relative mRNA expression of galectin-1 (LGALS1), galectin-3 (LGALS3), galectin-8 (LGALS8), semaphorin-3A (SEMA3A), semaphorin-3B (SEMA3B), semaphorin-3F (SEMA3F), neuropilin-1 (NRP1), neuropilin-2 (NRP2), VEGFA-D, VEGFR1-4, PDGFRu(PDGFRA), PDGFRβ (PDGFRB), KIT, E-cadherin (CDH1) and vimentine (VIM) in SQ20B cells. B—Western Blot of p-ERK1/2, p-AKTser473 and p-S6 on SQ20B cells treated with 100 ng/ml galectin-1 (Gall), 10 ng/ml semaphorin-3A (Sema3A) or 100 ng/ml VEGF165.

FIG. 8. OTX-008 effects in in vivo xenograft models. Images of Galectin-1, Ki67 (MIB-1), VEGFR2 and CD31 immunostaining on A2780-1A9 xenografts treated or not with OTX-008 (A) and on SQ20B, SQ20B treated with OTX-008 and SQ-shGAL1 xenografts (B).

### DETAILED DESCRIPTION

Disclosed herein are methods of inhibiting galectin-1, including galectin-1 activity and/or expression, including expression of galectin-1 mRNA and/or galectin-1 protein. Also disclosed herein are methods of inhibiting a galectin-1-associated pathway in a mammalian cell. Also disclosed herein are compositions and methods for treating cancer in mammals, via modulation of galectin-1. In an aspect, compositions and methods are provided for treatment of cancer in a mammal, including treatment of tumors, via modulation of galectin-1. In another aspect, the disclosure encompasses methods of using antiproliferative and anti-invasive effects of OTX-008 in cancer cells and human tumor xenografts to treat cancer or tumors. As demonstrated herein, OTX-008 yields its antiproliferative effects, in part, in cancer cells expressing low levels of galectin-1, high levels of epithelial markers such as E-cadherin, and low levels of mesenchymal markers such as vimentin. In an aspect, OTX-008 down-regulates MAPK and AKT/mTOR survival pathways and galectin-1 protein expression. In another aspect, induces MAPK-dependent G2/M-transition inhibition in vitro and inhibits tumor growth, angiogenesis, and metastasis in vivo. In an embodiment, encompassed herein are methods of targeting galectin-1 as a target for anticancer therapy. In an embodiment, encompassed herein are methods of using OTX-008 to target galectin-1 as a target for anticancer therapy. In an embodiment, encompassed herein are methods of treating cancers involving galectin-1-overexpressing cells. In an embodiment, encompassed herein are methods of treating cancers involving galectin-1-overexpressing cells by contacting galectin-1 overexpressing cells with OTX-008 in order to decrease or diminish the levels of galectin-1 mRNA and/or protein in such cells.

The synthesis, structure, properties, and activities of OTX-008, including antiangiogenic activity, are described in detail in U.S. Patent Publication No. 2008/0300164 A1 (Ser. No. 11/664,641; “the '641 application”), which is incorporated by reference herein in its entirety. OTX-008 is also referred to as “compound 40” and “KM0118” in the '641 application.

In an embodiment, OTX-008 has at least one of the biological activities described herein. The biological activity of a compound can be determined, for example, as described herein or by methods well known to one of skill in the art. As will be understood by the skilled artisan viewing the disclosure encompassed herein, an activity of OTX-008 is one which arises through modulation of galectin-1, as described herein.

In another embodiment, a galectin-1-targeting compound that is not OTX-008 is encompassed herein. In an embodiment, a composition is provided, the composition comprising at least two non-OTX-008 galectin-1-targeting compounds. In an embodiment, a composition is provided, the composition comprising OTX-008 and at least one non-OTX-008 galectin-1-targeting compound. In an aspect of an embodiment, the non-OTX-008 compound is a calixarene.

“Targeting” of galectin-1, as the term is used herein, encompasses the direct physical contact of a compound with—or affinity for—galectin-1, as well as any indirect effect of the compound on galectin-1, including down-regulation of galectin-1 expression via the compound's interaction with a non-galectin-1 moiety. OTX-008 “targeting” of galectin-1, as the term is used herein, encompasses the direct physical contact of OTX-008 with—or affinity for—galectin-1, as well as any indirect effect of OTX-008 on galectin-1, including down-regulation of galectin-1 expression via OTX-008 interaction with a non-galectin-1 moiety.

“Modulation” of galectin-1 or galectin-1 activity, as the term is used herein, encompasses the up- or down-regulation of galectin-1 expression, and the up- or down-regulation of galectin-1 activity, as well as causing galectin-1 to demonstrate a different, non-typical, or new activity or cellular role, whether or not the different, non-typical, or new activity or role also results in the up- or down-regulation of galectin-1 expression, or the up- or down-regulation of galectin-1 activity.

As the term is used herein, “galectin-1 activity” refers to the end results or processes resulting either directly or indirectly from the structure, location, catalysis (whether non-enzymatic or enzymatic), or intermolecular interactions of galectin-1 protein or nucleic acid. As disclosed in detail elsewhere herein, galectin-1 is involved in numerous inter- and intracellular metabolic pathways in various mammalian cells, and the role and function of galectin-1 in these metabolic pathways are encompassed by the term “galectin-1 activity”.

A “galectin-1-associated pathway” is any cellular or metabolic pathway involving galectin-1 protein or nucleic acid. For example, galectin-1-associated pathways include, but are not limited to, the galectin-1/semaphorin-3A system in cancer cells, pathways involving ERK1/2-, AKT-, and S6-phosphorylation, the MAPK pathway, and pathways involving p-wee1, among others. A galectin-1-associated pathway may be demonstrated, as disclosed in detail herein in the experimental examples, by in vivo or in vitro assays employing a known galectin-1-targeting compound. Assays which can be used to demonstrate a galectin-1-associated pathway include, but are not limited to, delay of tumor growth in A2780-1A9 human ovarian cancer and SQ20B human squamous cell head-and-neck cancer xenograft models, decreased galectin-1 protein expression and translocation of galectin-1 from the cytoplasm to the nucleus in vitro, and decreasing galectin-1 expression and decreasing Ki67 expression in vivo, in association with a demonstration of antiproliferative effects in cultured cells. The metabolic and signaling pathways involved in such assays are well-known.

“Treat”, “treating”, and “treatment”, etc., as used herein, refer to any action providing a benefit to a patient afflicted with a disease, including improvement in the condition through lessening or suppression of at least one symptom, delay in progression of the disease, prevention or delay in the onset of the disease, etc.

The term “pharmaceutically acceptable”, as used herein with respect to a compound or composition, refers to a form of the compound or composition that can increase or enhance the solubility or availability of the compound in a subject, in order to promote or enhance the bioavailability of the compound or composition. In an aspect, the disclosure herein also encompasses pharmaceutically acceptable, hydrates, solvates, stereoisomers, or amorphous solids of the compounds and compositions embodied herein.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 1990, incorporated herein by reference). Except insofar as any conventional carrier is incompatible with the active ingredient or method of use, its use in the pharmaceutical compositions is contemplated.

An “effective amount” of a galectin-1-targeting compound is an amount of the compound necessary to bring about a desired effect. For example, an effective amount of OTX-008 to inhibit growth or proliferation of human cancer cells, in vitro or in vivo, is an amount of compound required to observe inhibition of growth or proliferation of such cells, as compared to the growth or proliferation of such cells that are not contacted with OTX-008, but are otherwise identical to the cells contacted with OTX-008. It will be understood that “effective amount” therefore refers to low concentrations of compound which have a measureable but relatively small effect on the cells, as well as high concentrations of compound which have a measurable and substantial effect on the cells.

The term “therapeutically effective amount” is used herein, unless otherwise indicated, to describe an amount of a compound or composition which, in context, is used to produce or effect an intended therapeutic result. In an embodiment, the intended therapeutic result relates to the treatment of a hyperproliferative disease state, such as a tumor, including a carcinogenic tumor or other cancer or the treatment of a precancerous lesion or other cell(s) which express abnormal or foreign proteins or immunogens on a cell surface. With respect to an anticancer effect, that effect may be one or more of inhibiting further growth of tumor or cancer cells, inducing an antiangiogenic effect (e.g., by killing tumor endothelial cells), reducing the likelihood or eliminating metastasis or producing cell death in the tumor or cancer cells, resulting in a shrinkage of the tumor or a reduction in the number of cancer cells or preventing the regrowth of a tumor or cancer after the patient's tumor or cancer is in remission. As indicated, an anti-cancer agent may exhibit an anti-cancer effect alone and/or may enhance the ability of another anticancer agent to exhibit an anti-cancer effect. In an embodiment, the intended therapeutic result relates to the treatment of a hyperproliferative disease state that is a result or consequence of galectin-1 expression, function, or activity. In an embodiment, the intended therapeutic result is, at least in part, due to targeting of galectin-1 by a compound or composition. In an embodiment, targeting of galectin-1 involves physical interaction of galectin-1 and the compound or composition. In an embodiment, targeting of galectin-1 does not involve physical interaction of galectin-1 and the compound or composition, but nonetheless, involves a direct effect of the compound or composition on galectin-1, through one or more other moloecules. In an embodiment, targeting of galectin-1 involves both direct physical interaction with galectin-1 as well as one or more indirect interactions with galectin-1.

The terms “coadministration” and “combination therapy” are used to describe a therapy in which at least two compounds are used to treat cancer or another disease state or condition, as described herein, at the same time. In an embodiment, the terms “coadministration” and “combination therapy” are used to describe a therapy in which at least two compounds are used to treat cancer or another disease state or condition as described herein, at the same time, wherein one of the compounds is a galectin-1-targeting compound. In an embodiment, the terms “coadministration” and “combination therapy” are used to describe a therapy in which at least two compounds are used to treat cancer or another disease state or condition as described herein at the same time, wherein one of the compounds is OTX-008. In an embodiment, the activity of OTX-008 in a co-administered composition is mediated, at least in part, via modulation of galectin-1, the other compound or compounds in the co-administered therapy may or may not act via modulation of galectin-1. In an embodiment, combination therapy with OTX-008 is used to treat cancer or another disease state or condition, as described herein, at the same time. In another embodiment, a composition for combination therapy with OTX-008, which composition comprises an effective amount for treatment, is used to treat cancer or another disease state or condition, as described herein, at the same time. In an embodiment, the result of coadministration with OTX-008 may be additive of the treatment results obtained using each compound separately, either directly additive, or additive to a degree lesser than the results obtained with the two compounds separately. In an embodiment, the result of treatment via coadministration with OTX-008 may be synergistic, to varying degrees. In an embodiment, the result of treatment via coadministration with OTX-008 may be greater than the treatment results obtained using each compound separately. In an aspect, the result of treatment with a composition encompassed herein is such that, for one compound, the result of treatment is greater than that obtained with the compound separately, while the results of treatment with respect to the other compounds in the composition are about the same as the results of treatment obtained separately. In an aspect, the result of treatment for at least two compounds is greater than that obtained with the compounds separately, while the other compounds in the composition are about the same as the results of treatment obtained separately. In an aspect, the result of treatment for all compounds in the composition is greater than that obtained with the compounds separately. In an embodiment, the result of treatment via coadministration with OTX-008 may be less than the treatment results obtained using each compound separately. In an aspect, the result of treatment with a composition encompassed herein is such that, for one compound, the result of treatment is less than that obtained with the compound separately, while the results of treatment with respect to the other compounds in the composition are about the same as the results of treatment obtained separately. In an aspect, the result of treatment for at least two compounds is less than that obtained with the compounds separately, while the other compounds in the composition are about the same as the results of treatment obtained separately. In an aspect, the result of treatment for all compounds in the composition is less than that obtained with the compounds separately.

Treatment can be prophylactic or therapeutic. In an embodiment, treatment can be initiated before, during, or after the development of the cancerous condition. As such, the phrases “inhibition of” or “effective to inhibit” includes both prophylactic and therapeutic treatment, which includes prevention and/or reversal of the condition.

In some embodiments, although the term coadministration encompasses the administration of two active compounds to the patient at the same time, wherein the compounds are not administered to the patient at the same time, the effective amounts of the individual compounds will be present in the patient at the same time.

In an embodiment, a composition comprises a galectin-1-targeting compound with an optional carrier (e.g., a pharmaceutically acceptable carrier), which composition is added to cells in culture or used to treat a patient, such as a human. In an embodiment, a composition comprises OTX-008 with an optional carrier (e.g., a pharmaceutically acceptable carrier), which composition is added to cells in culture or used to treat a patient, such as a human. Where OTX-008 is used to treat a patient, it is preferably combined in a pharmaceutical composition with at least one pharmaceutically acceptable carrier, such as a larger molecule to promote stability, or with a pharmaceutically acceptable buffer that serves as a carrier.

OTX-008, or any other galectin-1-targeting compound, can be administered alone or in a pharmaceutically acceptable buffer, as an antigen in association with another protein, such as an immunostimulatory protein or with a protein carrier such as, but not limited to, keyhole limpet hemocyanin (KLH), bovine serum albumin (BSA), ovalbumin, or the like. It can also be used in adjuvant therapy, in combination with, for example, one or more chemotherapeutic agents like carboplatin or others known to one skilled in the art.

In an embodiment, a galectin-1-targeting compound, such as OTX-008, can be combined with a variety of physiological acceptable carriers for delivery to a patient including a variety of diluents or excipients known to those of ordinary skill in the art. For example, for parenteral administration, isotonic saline is preferred. For topical administration a cream, including a carrier such as dimethylsulfoxide (DMSO), or other agents typically found in topical creams that do not block or inhibit activity of the peptide, can be used. Other suitable carriers include, but are not limited to alcohol, phosphate buffered saline, and other balanced salt solutions.

In an embodiment, a composition comprising a compound disclosed herein, such as OTX-008, is cytotoxic to a cell. In an embodiment, the cell is a cancer cell. In an embodiment, the measure of cytotoxicity is obtained by contacting the cell with a compound disclosed herein and observing the effects on the cell using the MTT assay, as described in detail elsewhere herein, and comparing the observed effects with the appropriate controls containing untreated cells that are otherwise identical to the compound-treated cells. Cell parameters providing a measure of cytotoxicity include, but are not limited to, cell proliferation as a function of galectin-1 mRNA expression and/or galectin-1 protein expression.

Also provided herein is a method of identifying a cell susceptible to treatment with a galectin-1-targeting compound disclosed herein, such as OTX-008. In an embodiment, a galectin-1-targeting compound is cytotoxic to a cell susceptible to treatment with a galectin-1-targeting compound. In another embodiment, a galectin-1-targeting compound has an antiproliferative effect on such a cell. In another embodiment, galectin-1-targeting compound affects such a cell so that one or more metabolic or cell-cycle pathways in the cell are altered. In an embodiment, a cell described herein is a cancer cell. In an embodiment, a cell expressing a lower than normal level of galectin-1 will be suceptible to treatment with a galectin-1-targeting compound. In an embodiment, a cell expressing a higher than normal level of an epithelial marker will be susceptible to treatment with a galectin-1-targeting compound. In an embodiment, a cell expressing a higher than normal level of E-cadherin will be susceptible to treatment with a galectin-1-targeting compound. In another embodiment, an epithelial cell expressing a higher than normal level of keratin-8 and/or keratin-18 will be susceptible to treatment with a galectin-1-targeting compound. In an embodiment, a cell expressing a low level of a mesenchymal marker will be susceptible to treatment with a galectin-1-targeting compound. In an embodiment, a cell expressing a low level of vimentin will be susceptible to treatment with a galectin-1-targeting compound. In another embodiment, a cell having two or more of the characteristics described herein will be susceptible to treatment with a galectin-1-targeting compound. In an aspect, a method of predicting whether a cell will be susceptible to treatment with a galectin-1-targeting compound disclosed herein, such as OTX-008, comprises contacting the cell with a composition comprising a galectin-1-targeting compound. In another aspect, the composition is a pharmaceutical composition.

In an embodiment, a composition comprising OTX-008 is anti-angiogenic, via modulation of galectin-1. Angiogenesis is involved in numerous biological functions in the body, from normal processes like embryogenesis and wound healing to abnormal processes like tumor growth, arthritis, restenosis, atherosclerosis, diabetic retinopathy, neovascular glaucoma, and endometriosis. The use of agents that can inhibit angiogenesis in vitro and in vivo, particularly in anti-tumor research, has indicated that anti-angiogenic therapy is a promising therapeutic modality. The compositions and methods herein are useful for treating such processes and/or conditions in which galectin-1 plays a role.

In an embodiment, encompassed herein is a method for inhibiting endothelial cell proliferation in a patient (e.g., a mammal such as a human), via modulation of galectin-1. In an aspect, this encompasses administering to a patient an amount of a composition (typically a pharmaceutical composition) effective to inhibit the growth of endothelial cells, wherein the composition comprises OTX-008. In an embodiment, encompassed herein is a method for inhibiting endothelial cell proliferation in vitro (e.g., in a cell culture). In an aspect, this encompasses contacting cells with an amount of a composition effective to prevent and/or reduce the growth of endothelial cells, wherein the composition comprises OTX-008.

For determining the amount of endothelial cell proliferation in vivo, various methods known to one of skill in the art could be used, as demonstrated herein. For example, for evaluation of endothelial cell growth in tumors, tissue sections can be appropriately stained to quantify vessel density. For determining the amount of endothlelial cell proliferation in vitro, an Endothelial Cell Proliferation Assay can be used that involves the uptake of tritiated thymidine by cells in cell culture. In an embodiment, OTX-008 that is “active” for inhibiting endothelial cell proliferation is one that causes an at least 10% reduction in endothelial cell proliferation at a OTX-008 concentration lower than 10⁻⁴ M. Alternatively, inhibition of endothelial cell proliferation for an “active” OTX-008 in vitro is preferably at an IC50 level of less than 80 μM (more preferably less than 50 μM, and even more preferably less than 25 μM). In an embodiment, encompassed herein is a method for determining the amount of endothelial cell proliferation in vitro, wherein the method comprises an endothelial cell proliferation assay. In an embodiment, a measurement of inhibition of endothelial cell proliferation as described herein can be used to identify a therapeutically effective amount of a compound, such as, but not limited to, OTX-008. In an embodiment, encompassed herein is a method of correlating a therapeutically effective amount of a compound, as described elsewhere herein, with the amount of a compound considered to be active in the in vitro assays described herein.

In an embodiment, encompassed herein is a method for inhibiting angiogenic-factor mediated intercellular adhesion molecule (ICAM) expression down-regulation (and/or promoting ICAM expression) in a patient (e.g., a mammal such as a human), via modulation of galectin-1. In an embodiment, this involves administering to a patient an amount of a composition effective to prevent and/or reduce the amount of ICAM expression down-regulation, wherein the composition comprises OTX-008 and functions via modulation of galectin-1. In an embodiment, the amount of a compound, whether alone or as part of a composition, effective to prevent and/or reduce the amount of ICAM expression down-regulation is a therapeutically-effective amount, as described elsewhere herein. Analogously, the present invention provides a method for inhibiting angiogenic-factor mediated intercellular adhesion molecule expression down-regulation (and/or promoting ICAM expression) in vitro (e.g., in a cell culture). This method involves contacting cells with an amount of a composition effective to prevent and/or reduce the amount of ICAM expression down-regulation, wherein the composition comprises OTX-008. In an embodiment, expression of ICAM, such as ICAM-1 protein, can be examined by flow cytometry with R-phycoerythrin-conjugated anti-ICAM-1 (CD54) monoclonal antibody or mouse IgGl isotype control. In another embodiment, expression of ICAM nucleic acid can be monitored for up- or down-regulation. It will be understood by the skilled artisan, when armed with the disclosure set forth herein, that the nucleic acid expression patterns may differ from protein levels of ICAM expressed, even when based on the same sample and measured at the same time. The present disclosure provides the skilled artisan with the guidance necessary to correlate in vitro results with the results obtained upon treatment of a mammal with a compound or composition set forth herein, so that a therapeutically effective amount of a compound or composition can be correlated with the in vitro assay using the compound or composition.

In an embodiment, a method is provided for increasing the infiltration of leukocytes into tumor tissue in a patient (e.g., a mammal such as a human). In an embodiment, this involves administering to a patient an amount of a composition effective to increase the amount of white blood cells (leukocytes) that can infiltrate into the tumor tissue through blood vessels via modulation of galectin-1, wherein the composition comprises OTX-008. The use of agents that can increase leukocyte infiltration into tumor tissue, particularly in anti-tumor research, has been sought for some time and in the general area of immunotherapy and will be a promising therapeutic modality in the future.

In an embodiment, a method is provided for inhibiting angiogenesis (i.e., new blood vessel formation) in a patient (e.g., a mammal, such as a human). In an embodiment, this involves administering to a patient an amount of a composition effective to prevent and/or reduce angiogenesis, via galectin-1 modulation, wherein the composition comprises OTX-008. In another embodiment, a method is provided for inhibiting angiogenesis in vitro (e.g., in a cell culture). This method involves contacting cells with an amount of a composition effective to prevent and/or reduce angiogenesis via modulation of galectin-1, wherein the composition comprises OTX-008.

For determining the amount of angiogenesis in vivo, various methods known to one of skill in the art could be used. For example, for evaluation of angiogenesis in tumors, tissue sections can be appropriately stained to quantify vessel density. For determining the amount of angiogenesis in vitro, an angiogenesis assay can be used that involves the disappearance of endothelial cell sprouting in cell culture. In an embodiment, a polypeptide that is “active” for angiogenesis inhibition is preferably one that causes an at least 10% reduction in endothelial cell sprouting at a concentration lower than 10⁻⁴ M. In another embodiment, inhibition of angiogenesis for a composition comprising OTX-008 in vitro is preferably at a level of less than 85% sprouting (more preferably less than 75% sprouting, even more preferably 50% sprouting, and even more preferably less than 35%) as determined using a collagen gel-based assay as known in the art, such as that described in detail in U.S. Patent Publication No. 2008/0300164 A1 (Ser. No. 11/664,641), which is incorporated by reference herein in its entirety.

In an embodiment, a method is provided for inhibiting tumorigenesis in a patient (e.g., a mammal such as a human) via modulation of galectin-1. In an embodiment, the method comprises administering to a patient an amount of a composition effective to prevent and/or reduce tumor growth, via modulation of galectin-1, wherein the composition comprises OTX-008. Methods of determining the inhibition of tumorigenesis are well known to those of skill in the art, including evaluation of tumor shrinkage, survival, etc. In an embodiment a method is provided for treatment of cancer in a patient, wherein the treatment of the cancer includes preventing or slowing the growth of the cancer. In an embodiment a method is provided for treatment of cancer in a patient, wherein the treatment of the cancer includes preventing the spread of a tumor associated with the cancer. In an embodiment a method is provided for treatment of cancer in a patient, wherein the treatment of the cancer includes preventing the spread of one or more metastases associated with the cancer. In an embodiment a method is provided for treatment of cancer in a patient, wherein the treatment of the cancer includes reducing the size of a tumor associated with the cancer. In an embodiment a method is provided for treatment of cancer in a patient, wherein the treatment of the cancer includes preventing the recurrence of cancer treated previously.

As will be understood based on the disclosure set forth herein, cancers in which galectin-1 is believed to play a role are cancers that may be treated using the compositions and methods set forth herein. Such cancers include, but are not limited to, ovarian cancer, squamous cell carcinoma, cancers of the digestive system, including stomach, liver, and colon cancer, cancers of the thyroid, cancers of the endometrium and ovaries, including adenocarcinoma of the endometrium, uterine cancers, including uterine adenocarcinomas and uterine smooth muscle tumors, breast cancer, prostate cancer, bladder cancer, head and neck cancers, including gliomas, kidney cancers, pancreatic cancer, including pancreatic ductal adenocarcinoma, nonsmall-cell lung cancer, and melanoma.

In an embodiment, a method is provided for treatment of a cancer in a patient, wherein the cancer is associated with overexpression of galectin-1. As will be understood by the skilled artisan, overexpression of galectin-1 encompasses levels of expression of galectin-1 exceeding typical, normal, or “healthy” levels of galectin-1 expression. In an embodiment, the method comprises administering to a patient a therapeutically effective amount of a galectin-1-targeting composition effective to treat the cancer in a patient, wherein the cancer is associated with overexpression of galectin-1. In an embodiment, the method comprises administering to a patient a therapeutically effective amount of OTX-008 effective to treat the cancer in a patient, wherein the cancer is associated with overexpression of galectin-1. In an embodiment, the cancer cells overexpress galectin-1. In another embodiment, the cancer cells do not overexpress galectin-1, but at least one non-cancerous cell in the patient overexpresses galectin-1, wherein the non-cancerous cell is responsible, at least in part, for the cancer. In another embodiment, the cancer cells overexpress galectin-1, and at least one non-cancerous cell in the patient overexpresses galectin-1, wherein the non-cancerous cell is responsible, at least in part, for the cancer. In an aspect of an embodiment, a galectin-1-targeting composition, such as a composition comprising OTX-008, when administered to a patient, contacts at least one cancer cell in the patient. In another aspect of an embodiment, a galectin-1-targeting composition, such as a composition comprising OTX-008, when administered to a patient, contacts at least one cancer cell in the patient, wherein the cancer cell overexpresses galectin-1.

Compositions useful for treatment as described herein include pharmaceutically acceptable salts of the compounds in the composition, including OTX-008. As used herein, the term pharmaceutically acceptable salts or complexes refers to salts or complexes that retain the desired biological activity of the parent compound and exhibit minimal, if any, undesired toxicological effects. While it will be understood that a peptidomimetic such as OTX-008 may practically form different useful salts than a small molecule drug, nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acids, naphthalenedisulfonic acids, and polygalacturonic acid; (b) base addition salts formed with polyvalent metal cations such as zinc, calcium, bismuth, barium, magnesium, aluminum, copper, cobalt, nickel, cadmium, sodium, potassium, and the like, or with an organic cation formed from N,N-dibenzylethylene-diamine, ammonium, or ethylenediamine; or (c) combinations of (a) and (b); e.g., a zinc tannate salt or the like.

Modifications of a compound can affect the solubility, bioavailability and rate of metabolism of the active species, thus providing control over the delivery of the active species. Further, the modifications can affect the anticancer activity of the compound, in some cases increasing the activity over the parent compound. This can easily be assessed by preparing the derivative and testing its anticancer activity according to the methods encompassed herein, or other methods known to those skilled in the art.

Compositions encompassed herein may be administered to mammals parenterally, by inhalation spray, rectally, nasally, buccally, vaginally or via an implanted reservoir. The term “parenteral” as used herein includes intradermal, subcutaneous, intravenous, intramuscular, intra-articular, intra-synovial, intrasternal, intrathecal, intrahepatic, intralesional and intracranial injection or infusion techniques.

In an embodiment, a composition is formulated to independently include between about 0.1 milligram and about 2000 milligrams of each active compound, including anti-cancer compounds, anti-angiogenic compounds, and anti-emetic compounds, among others, as well as bioactive compounds. In an embodiment, a composition is formulated to independently include any value between about 0.1 milligram and about 2000 milligrams of each active compound, including anti-cancer compounds, anti-angiogenic compounds, and anti-emetic compounds, among others, as well as bioactive compounds. In an embodiment, a composition is formulated to independently include any value between about 0.1 milligram and about 2000 milligrams of each active compound per unit dosage form, including anti-cancer compounds, anti-angiogenic compounds, and anti-emetic compounds, among others, as well as bioactive compounds. It will be understood that the solubility limitations of any compound, including OTX-008, will be taken into consideration when developing dosage forms and dosing regimens.

In an embodiment, compositions comprising a galectin-1-targeting compound, such as OTX-008, can be administered as a single dose or in multiple doses. Preferably the dose is an effective amount as determine by the standard methods described herein and includes about 1 microgram to about 1,000 micrograms pretreatment, about 25 micrograms to about 500 micrograms pretreatment, about 50 to about 250 micrograms pretreatment, about 2 micrograms to about 100 micrograms pretreatment, about 5 micrograms to about 75 micrograms pretreatment, about 10 micrograms to about 50 micrograms pretreatment, about 20 micrograms to about 40 micrograms pretreatment, and as discussed in greater detail elsewhere herein. Those skilled in the art of clinical trials will be able to optimize dosages of OTX-008-containing compositions through standard trial studies, in view of the disclosure encompassed herein.

In an embodiment, OTX-008, and the therapeutic composition as encompassed herein, is included in a pharmaceutically acceptable carrier or diluent in an amount sufficient to deliver to a patient a therapeutically effective amount for the desired indication, without causing serious toxic effects in the patient treated. In an embodiment, OTX-008 is administered to a patient in the range of about 1 mg/day to about 2000 mg/day. In another embodiment, OTX-008 is administered to a patient in the range of about 5 mg/day to about 1500 mg/day, about 10 mg/day to about 1250 mg/day, about 20 mg/day to about 1000 mg/day, about 30 mg/day to about 750 mg/day, about 40 mg/day to about 500 mg/day, about 50 mg/day to about 250 mg/day, and about 60 mg/day to about 100 mg/day. In an embodiment, OTX-008 is administered to a patient in the range of about 500 mg/day to about 600 mg/day. In an embodiment, OTX-008 is administered to a patient in a dose of about 65 mg/day. In another embodiment, a dose of the active compound for all of the herein-mentioned conditions is in the range from about 10 ng/kg to about 300 mg per kilogram body weight of the recipient/patient per day, from about 0.1 mg/kg to about 100 mg/kg per day, from about 0.5 mg/kg to about 25 mg/kg per day. The compound is conveniently administered in any suitable unit dosage form.

Unless otherwise specified to the contrary, the methods encompassed herein should be understood to embody the use of a compound encompassed herein, a composition comprising a compound encompassed herein, or a composition comprising at least two compounds disclosed herein. As will be understood from the present disclosure, a composition comprising one or more compounds may be a pharmaceutically-acceptable composition, or other composition including at least one substance in addition to the compound.

It will be understood, based on the disclosure set forth herein, in view of the skill in the art, that specific dosage for compounds and compositions encompassed herein may be determined empirically through clinical and/or pharmacokinetic experimentation, and that such dosages may be adjusted according to prespecified toxicity criteria. It will also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

Compounds and compositions encompassed herein, or derivatives of the same, can be prepared according to the methods encompassed herein and/or known the art.

In an embodiment, the compositions encompassed herein are administered by way of a single dosage form. In another embodiment, the compositions are administered in multiple dosage forms. In an aspect, different agents are administered in different dosage forms.

In an embodiment, a method for treating cancer comprises administering to a mammal in need thereof a therapeutically effective amount of a composition comprising at least two anti-cancer agents, or pharmaceutically acceptable salts, hydrates, solvates, stereoisomers, or amorphous solids thereof, wherein one of the compounds is OTX-008, and the composition acts, at least in part, via modulation of galectin-1. In an embodiment, slowing or ceasing tumor progression is a sign of treatment of cancer (e.g., in advanced and/or metastatic cancer).

In an embodiment, disclosed herein are methods of treating a subject that can benefit from administration of a galectin-1-targeting compound. In an aspect, the subject is a human patient. In an embodiment, a method of treating cancer in a patient having cancer includes contacting one or more cancerous cells in the subject with a therapeutically effective amount of a galectin-1 targeting compound. In an embodiment, the compound is OTX-008. In an embodiment, a method of inhibiting growth and/or proliferation of a cancer cell includes contacting a cancer cell with a therapeutically effective amount of a galectin-1 targeting compound. In an embodiment, the compound is OTX-008. In an embodiment, a method of inhibiting tumor growth and/or metastasis in a patient having a tumor includes contacting a tumor with a therapeutically effective amount of a galectin-1 targeting compound. In an embodiment, the compound is OTX-008. In an embodiment, a method of inhibiting galectin-1 activity and/or expression includes contacting a cell with a therapeutically effective amount of a galectin-1 targeting compound. In an embodiment, the compound is OTX-008.

In an embodiment, a method of treating a patient includes administration of a composition encompassed herein to treat cancer, and the treatment of the cancer includes at least one of preventing or slowing the growth of the cancer, preventing the spread of a tumor associated with the cancer, preventing the spread of one or more metastases associated with the cancer, reducing the size of a tumor associated with the cancer, and preventing the recurrence of cancer which was treated previously, wherein galectin-1 plays a role in the progression of the cancer. In an aspect, an administered composition comprises OTX-008, and the composition acts, at least in part, via modulation of galectin-1.

A subject suffering from cancer can be treated by administering to the subject an effective amount of a composition comprising OTX-008 as encompassed herein, optionally in a pharmaceutically acceptable carrier or diluent, either alone, or in combination with other known anticancer or pharmaceutical agents. This treatment can also be administered in conjunction with other conventional cancer therapies, such as radiation treatment, chemotherapy, or surgery.

In an embodiment, a therapeutic composition is administered on a schedule once a day. In an embodiment, a therapeutic composition is administered twice a day. In an embodiment, a therapeutic composition is administered three times a day, four times a day, five times a day, or more. In an embodiment, a therapeutic composition is administered less frequently than once a day. In an embodiment, a therapeutic composition is administered once every two days, once every three days, once every four days, once every five days, once every six days, or once every seven days. In an embodiment, a therapeutic composition is administered less frequently than once a week. In an embodiment, a therapeutic composition is administered once a month. In an embodiment, a therapeutic composition is administered twice a month.

In an aspect, based on the disclosure set forth herein, the skilled artisan will understand how to modify the compounds included in the composition, how to add or remove specific compounds from the composition, and how to adjust the dosage amounts, the dosage frequency, and the route of administration in order to optimize the treatment for a specific subject having a specific type of cancer or cancers. The concentration of active compounds in the composition will depend on absorption, distribution, inactivation, and excretion rates of the compound, as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the individual administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Embodiments of the present disclosure are further described by the following examples. It should be recognized that variations based on the inventive features are within the skill of the ordinary artisan, and that the scope of the disclosure herein should not be limited by the examples. To properly determine the scope of the present disclosure, an interested party should consider the claims herein, and any equivalent thereof. All patents, patent applications, and references cited herein are hereby incorporated by reference in their entirety.

### EXAMPLES

Materials.

OTX-008 was supplied by Oncoethix (Lausanne, Switzerland). UO126 was purchased from Sigma (Saint Quentin Fallavier, France). Recombinant galectin-1, semaphorin-3A and Neuropilin-1 were purchased from R&D Systems (Minneapolis, Minn., USA). Lactose was purchased from VWR (Fontenay-sous-Bois, France) and VEGF165 from Humanzyme (Chicago, Ill., USA).

Cell lines.

SCC61, SKBR3, MCF7, SKOV3, HEP2, SQ20B, CAKI1, HT29, OVCAR3, DU145, IGROV1 and PC3 cell lines were obtained from the ATCC (Rockville, Md., USA). HCT116, COL0205, HCC2998, HOP62 and HOP92 cell lines were obtained from the National Cancer Institute collection. A2780-1A9 cell line was obtained from the institute Mario Negri, Italy. DLD-1TR21-hSnail colon cancer cell line, which contains inducible SNAIL, and MCF7-shWISP2 (with WISP2 knockdown), were kindly provided by Geert Berx (Ghent, Belgium) and Michele Sabbah, Saint-Antoine Hospital (Paris, France), respectively. COL0205-R cells were developed in-laboratory. Cells were grown as monolayers in RPMI medium supplemented with 10% fetal calf serum (Invitrogen, Cergy-Pontoise, France), 2 mM glutamine, 100 units/ml penicillin and 100 μg/ml streptomycin at 37° C. in a humidified 5% CO₂ atmosphere, and regularly checked for the absence of Mycoplasma.

Cell Cytotoxicity Assay.

Cell survival was determined using the MTT assay (3-[4,5-dimethylthiazol-2-yl]-2,5-diphenyltetrazolium bromide; Sigma, Saint-Quentin Fallavier, France). The conversion of yellow water-soluble tetrazolium MTT into purple insoluble formazan is catalyzed by mitochondrial dehydrogenases and used to estimate the number of viable cells. In brief, cells were seeded in 96-well tissue culture plates at a density of 2×10³ cells/well. After 72 h drug exposure and 48 h wash-out, cells were incubated with 0.4 mg/ml MTT for 4 hours at 37° C. After incubation, the supernatant was discarded, insoluble formazan precipitates were dissolved in 0.1 ml of DMSO and the absorbance was measured at 560 nm by use of a microplate reader (Thermo, France). Wells with untreated cells or with drug-containing medium without cells were used as positive and negative controls respectively. IC₅₀'s were determined as the halves of maximal inhibitory drug concentration for each cell line.

In Vitro Growth Inhibition Assay.

For proliferation assay, cells were seeded in 96-well tissue culture plates at a density of 1.5×10³ cells/well. MTT assay was conducted daily to determine the number of viable cells in untreated control and treated group. Growth inhibition curves were plotted as a percentage of cells for each experimental condition.

Real-Time RT-PCR.

The theoretical and practical aspects of real-time quantitative RT-PCR using the ABI Prism 7900 Sequence Detection System (Perkin-Elmer Applied Biosystems, Foster City, Calif., USA) have been described in detail elsewhere Results were expressed as n-fold differences in target gene expression relative to the TBP gene (an endogenous RNA control) and relative to a calibrator (1× sample), consisting of the cell line sample from our tested series that contained the smallest amount of target gene mRNA. Experiments were performed in duplicate.

Western Blot Analysis.

Cells were lysed in buffer containing 50 mM HEPES (pH 7.6), 150 mM NaCl, 1% Triton X-100, 2 mM sodium vanadate, 100 mM NaF, and 0.4 mg/ml phenylmethylsulfonyl fluoride. Equal amounts of protein (30 μg/lane) were subjected to SDS-PAGE and transferred to nitrocellulose membranes. Membranes were blocked with 5% milk in 0.05% Tween 20/phosphate-buffered saline and then incubated with the primary antibody overnight. Membranes were then washed and incubated with the secondary antibody conjugated to horseradish peroxidase. Bands were visualized by using the enhanced chemiluminescence Western blotting detection system. Densitometric analysis was performed under conditions that yielded a linear response. The following antibodies were used: anti-p-cdc2 (tyr15), anti-p-wee1 (ser642), anti-p-p44/42 MAPK (thr202/tyr204), anti-p44/42, anti-p-AKT (ser473), anti-AKT, anti-p-S6 (ser235/236) (Cell Signaling, Saint Quentin Yvelines, France), anti-p-PKCα (ser657) (BD Biosciences, Le-Pont-de-Claix, France) and anti-galectin-1 (Abcam, Cambridge, Mass., USA). All antibodies were used at a 1:1000 dilution.

Cell Cycle Analysis.

Cell cycle analysis was assessed by flow cytometry. In brief, cells were seeded onto 25 cm³ flasks and treated with various concentrations of OTX-008. After 72 h exposure, adherent and non-adherent cells were recovered, washed with PBS, fixed in 70% ethanol and stored at −20° C. until use. Cells were rehydrated in PBS, incubated for 30 minutes at 37° C. with 200 μg/ml RNAse A, and for 20 minutes at +4° C. with 40 μg/ml propidium iodide in the dark. The cell cycle distribution was determined with a flow cytometer (FACSCalibur and Cell Quest Pro software, BD Biosciences, Le-Pont-de-Claix, France).

Immunohistochemistry.

The immunohistochemical procedure was performed on OCT-embedded subcutaneous tumor stained with H&E, MIB1 ( 1/400, Dako, Glostrup, Denmark), CD31 ( 1/200, Dako), VEGFR2 ( 1/125, Cell Signaling, Saint Quentin Yvelines, France) or galectin-1 (1:20, Abcam, Cambridge, Mass., USA) using an automated immunohistochemical stainer according to the manufacturer's guidelines (streptavidin-peroxidase protocol; Benchmark, Ventana, Tuscon, Ariz., USA). The images were captured and analyzed with a Zeiss observer Z1 microscope. Images quantification was performed using Histolab Software (Microvision, France) subtracting the background. Magnification ×20 and ×40.

Immunofluorescence.

The cells were grown on cover slips at 37° C. overnight. The growth media was then removed; the cells were washed with PBS and fixed in 4% of paraformaldhyde for 15 min and permeabilized with PBS/tritonX100 (1:500) then washed again with PBS. Incubation with primary antibodies was performed at 4° C. overnight (galectin-1, 1:100; Abcam, Cambridge, Mass., USA), followed by the incubation with the secondary antibodies (anti-mouse, 1:100; Cell Signaling, Saint Quentin Yvelines, France) for 1 h at room temperature in the dark. The nuclei were stained with Dapi 1:10000 (Santa Cruz Biotechnology, Santa Cruz, Calif., USA). The images were captured and analyzed with a Zeiss observer Z1 microscope. Images quantification was performed using Histolab Software (Microvision, France) subtracting the background. Magnification ×20 and ×40.

Small Hairpin RNA.

Galectin-1 shRNA, Semaphorin-3A shRNA and scrambled shRNA plasmids were purchased from Santa Cruz Biotechnology (Santa Cruz, Calif., USA) and transfected into cells using the manufacturer's shRNA transfection protocol. Briefly, a mix of 1 μg shRNA plasmid DNA solution and lpl shRNA plasmid transfection reagent were incubated 45 minutes at room temperature and then added on 50% confluent cells with shRNA plasmid transfection medium. After 6 hours incubation at 37° C., normal growth medium containing double serum and antibiotics concentration was added. After 24 h, the stably transfected cells were selected with puromycin.

Matrigel Invasion Assay.

8 μm membranes were coated with Matrigel (50 μg). The inserts were placed within a 24-well chamber containing 0.6 ml of RPMI 1640 medium with 20% fetal bovine serum as chemoattractant. 10⁵ cells were seeded into the inserts suspended in 0.3 ml of serum-free RPMI. After incubation for 48 h at 37° C., the upper surface of the filter was scraped to remove non-invasive cells. Invasive cells were fixed and stained with a Diff Quik detection Kit (Dade Behring, Paris, France). For quantification, the average number of invading cells per field was assessed by counting 9 random fields under a light microscope (400×).

A2780-1A9 Xenograft Models.

A volume containing 8.10⁶ A2780-1A9 cancer cells was injected s.c. into the right lateral flank of female nu/nu athymic mice. When tumors become palpable, mice were randomized and treated i.p. with PBS (3 days per week), 5 mg/kg OTX-008 (3 days per week), 6 mg/kg cisplatin (days 1, 8 and 15) or 10 mg/kg taxotere (days 1, 8 and 15). Tumor size was measured three times per week with caliper and tumor volume was calculated as (length×width²)/2. At the end of the study, animals were sacrificed and tumors collected and frozen in OCT for molecular biology and immunohistochemical analysis. Mouse experiments were approved by the Animal Housing and Experiment Board of the French government.

SQ20B Xenograft Models.

5.10⁶ SQ20B or SQ20B-shGal1 cancer cells were injected s.c. into the right lateral flank of female nu/nu athymic mice. On day 5, mice were randomized; one group of SQ20B-mice was treated every other day with 10 mg/kg OTX-008 i.p. Tumor size was measured twice weekly with caliper and tumor volume was calculated as 3.14(width²)/length. At the end of the study, mice were sacrificed, tumors were weighed and stored in OCT for molecular biology and immunohistochemical analysis. Secondary tumors were analyzed on liver, lung, intestine and skin in all mice. Mouse experiments complied with guidelines for Animal Housing and Experiment Board of the French authorities.

Antiproliferative Effects of OTX-008 Correlate with Galectin-1 Expression.

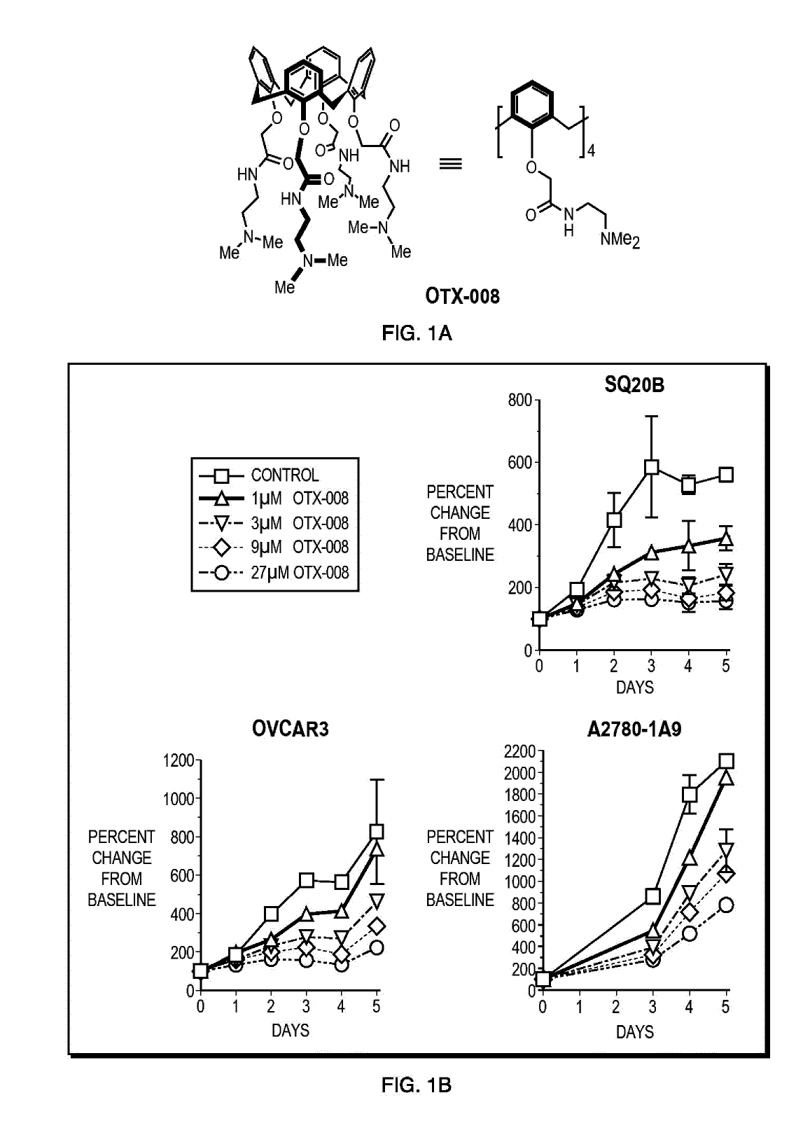

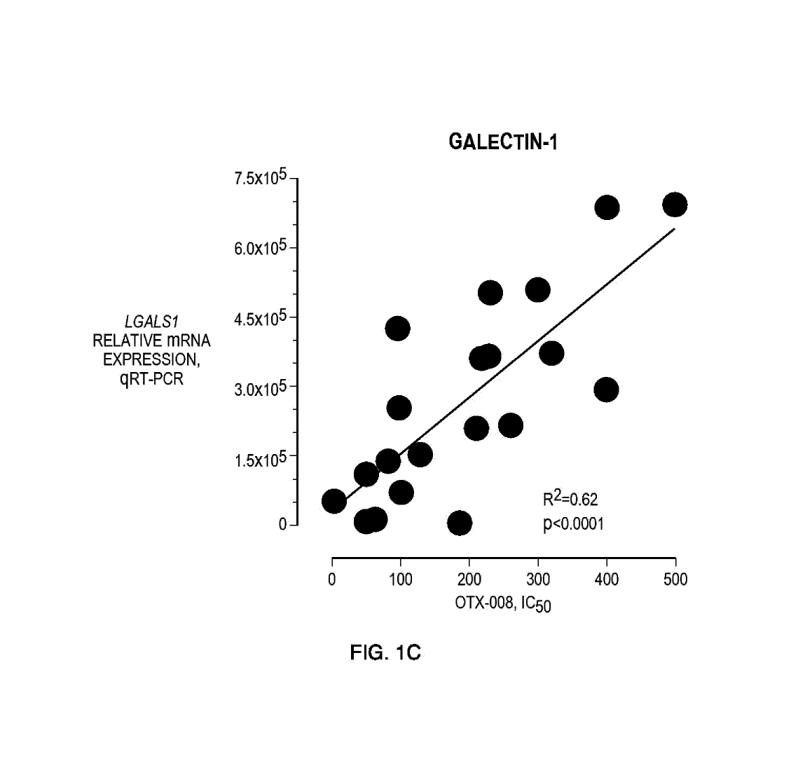

The antiproliferative effects of OTX-008 and anginex were assessed using MTT assay in a panel of human cancer cell lines of human origins expressing various levels of Lgals-1 mRNA and galectin-1 protein expressions (Supplementary Table 1). While anginex displayed limited effects in cultured cancer cells (Supplementary Table 1), OTX-008 showed dose-dependent antiproliferative effects in SQ20B, OVCAR3, and A2780-1A9, the later being only marginally sensitive to OTX-008 (FIG. 1B). Remaining cell lines were less sensitive to OTX-008 with GI₅₀ values >9 μM. OTX-008 displays no antiproliferative effect in cultured fibroblasts. Interestingly, the antiproliferative effects of OTX-008 in cancer cells were inversely correlated with GAL-1 mRNA (FIG. 1C) and protein expression. No correlation between mRNA expressions of galectin-2, galectin-3, and galectin-8 as well as semaphorin-3A, semaphorin-3B, neuropilin-1, VEGFR1-3, VEGFA-D, and PDGFRa&P3 and sensitivity to OTX-008 were detected in the panel.

Epithelial-to-Mesenchymal Transition Affects OTX-008 Sensitivity In Vitro.

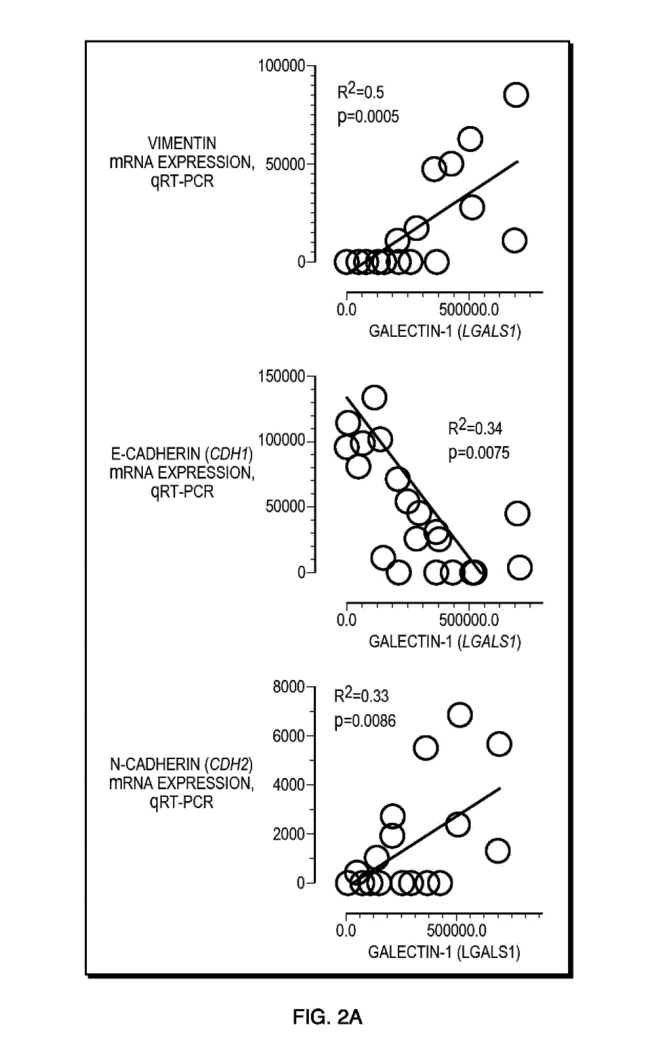

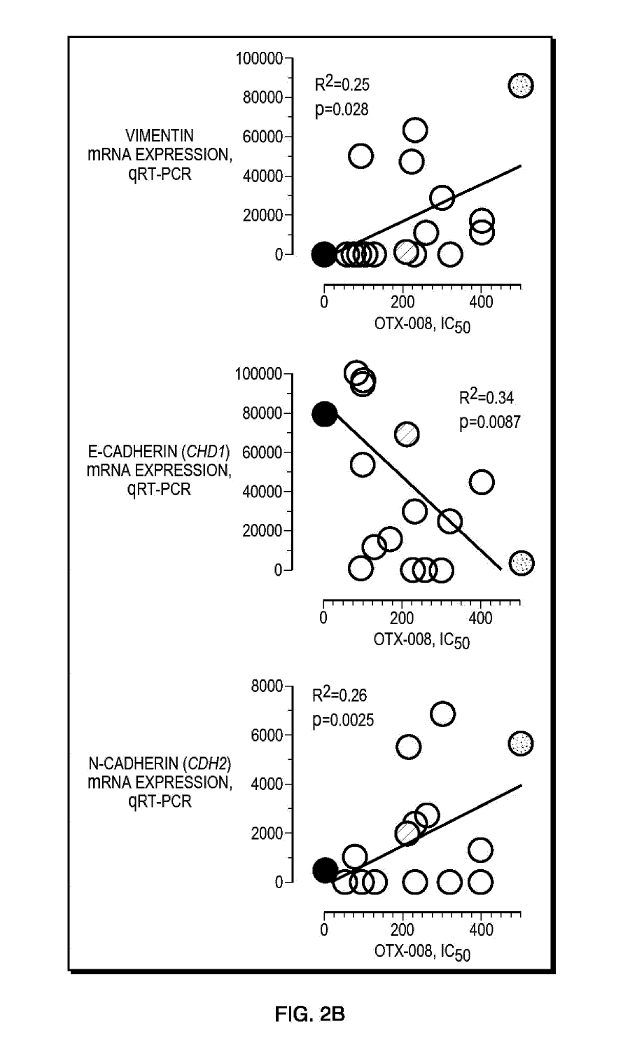

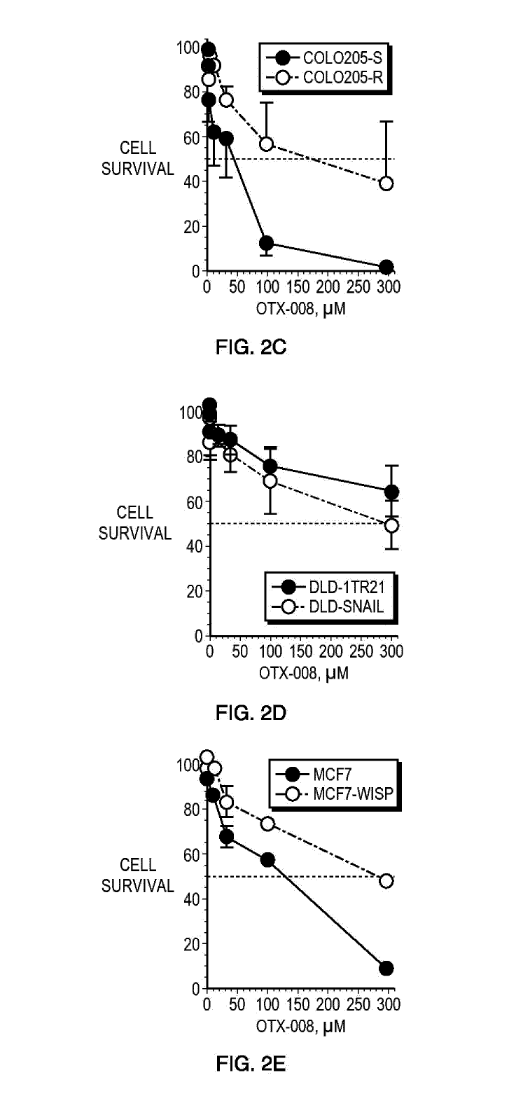

Galectin-1 expression has been associated with cell invasion and migration properties that are usually observed during epithelial-to-mesenchymal differentiation. As shown in FIG. 2A, high mRNA expression of galectin-1 (Lgals-1) strongly correlated with a mesenchymal phenotype as evaluated in the panel of cancer cells by the high vimentin (VIM), high N-cadherin (CDH2), and low E-cadherin (CDH1) mRNA expressions. In the study, the more OTX-008 sensitive SQ20B also expressed an epithelial phenotype. Further correlated in the cellular panel were the antiproliferative effects of OTX-008 with E-cadherin and vimentin mRNA expression (FIG. 2B). Consistently, cancer cells expressing mesenchymal markers such as vimentin were markedly more resistant to OTX-008 while high E-cadherin mRNA expressing cells appear to be more sensitive to the antiproliferative effects of OTX-008. Mesenchymal cancer cells resistant to OTX-008 expressed high mRNA FGFR1 while epithelial cancer cells more sensitive to OTX-008 expressed other epithelial markers such as keratin-8 (KRT8) and keratin-18 (KRT18). To characterize the mechanisms associated with epithelial-to-mesenchymal transition in cancer cells resistant to OTX-008, transcription factor expressions including SNAIL, SLUG, TWIST, SIP1, ZEB1, ACTA2, and HMGA2 as well as markers of main molecular pathways such as N-cadherin, TCF3, claudin-4 (CLDN4) were investigated, as were endothelin-1 (EDTI) mRNA levels. It was found that SNAIL, SLUG, SIP1 were randomly distributed while high mRNA expressions of ZEB1 and TWIST along with high N-cadherin and endothelin-1 and/or low claudin-4 were associated with resistance to OTX-008. Previously developed was COL0205-R, a cell line with strong mesenchymal differentiation from the parental epithelial COL0205-S cell line. It was shown (FIG. 2C) that OTX-008 was markedly more active in COL0205-S cells than in COL0205-R cells, supporting the importance of epithelial differentiation in sensitivity to OTX-008. OTX-008 was also tested in SNAIL-inducible model in DLD-1TR21 colon cancer cells (FIG. 2D) and in MCF7-shWISP (FIG. 2E), two other models of EMT. The latter model was obtained by transfection of shRNA WISP in MCF7 breast cancer cells which confers mesenchymal phenotype. As shown in FIGS. 2D and 2E, induction of SNAIL has no impact on sensitivity to OTX-008 although inhibition of WISP was associated with marked resistance to high concentrations of the drug.

Effects of OTX-008 on Intracellular Galectin-1 Expression.

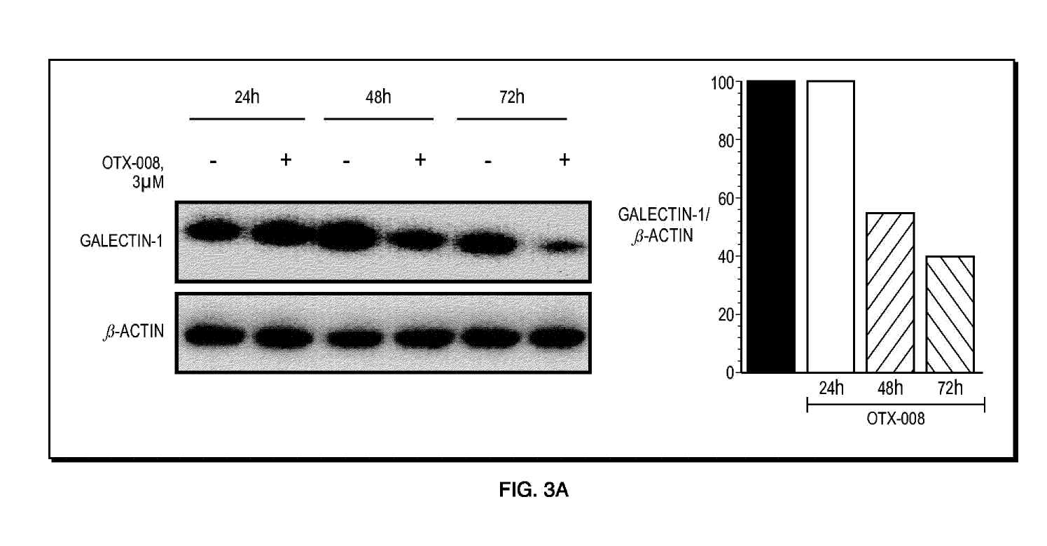

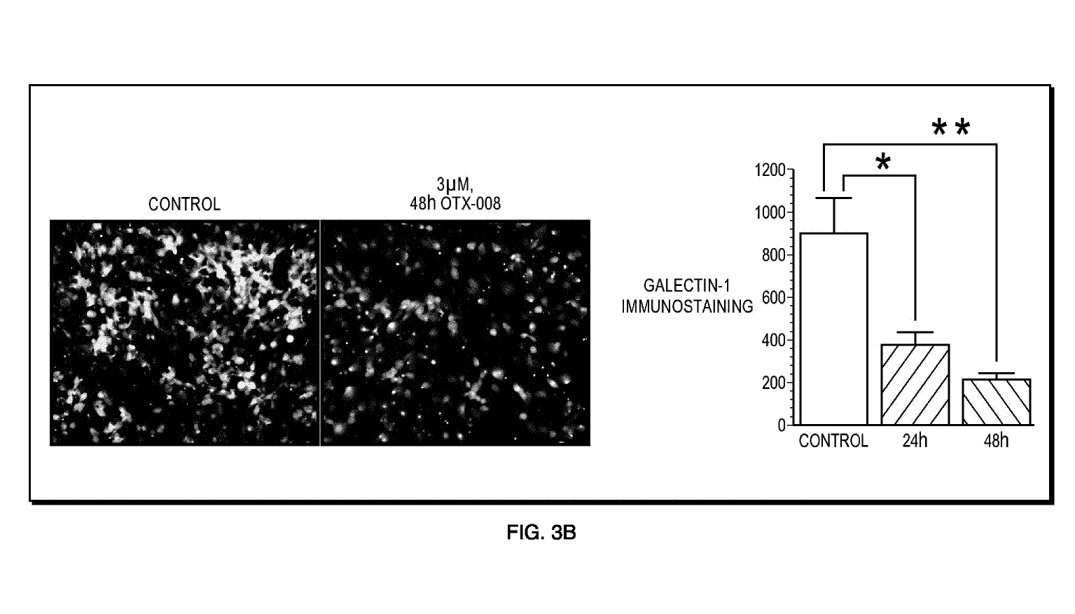

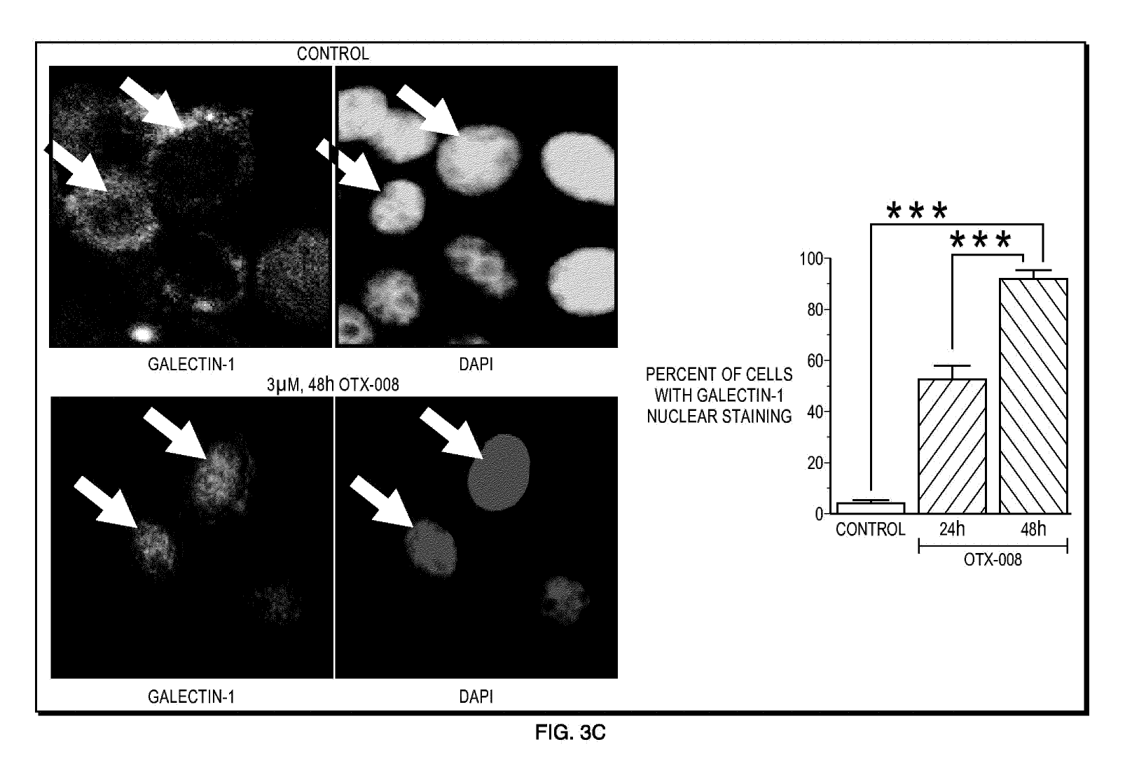

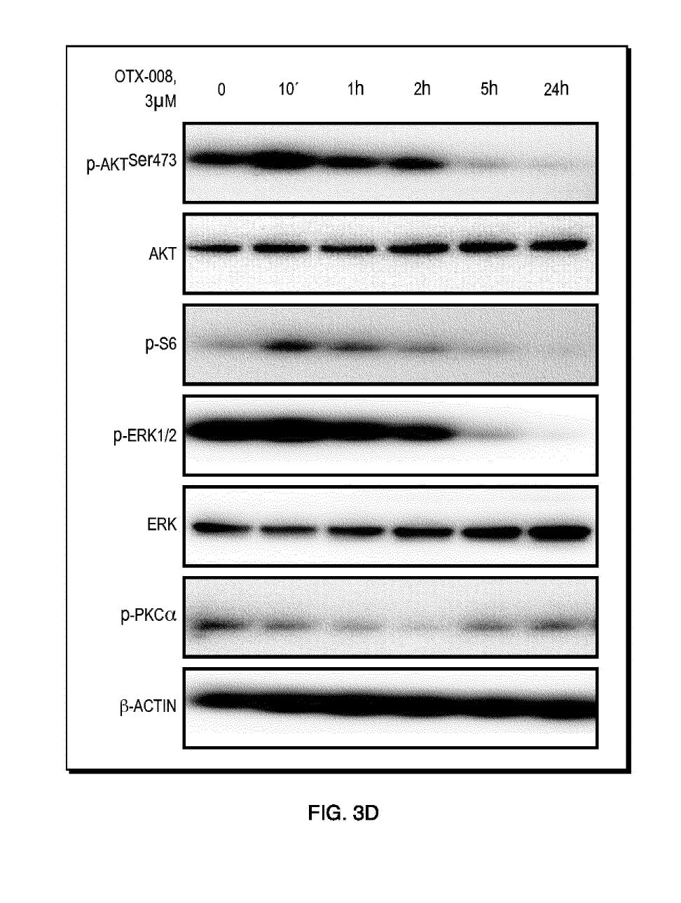

Immunohistochemistry showed that galectin-1 expression could be observed in the cytoplasm and the plasma membrane of SQ20B cells (FIG. 3A). OTX-008 treatment decreased galectin-1 protein expression in SQ20B cells after 48 and 72 h exposure (FIG. 3B). Similar results were obtained after 72 h exposure of A2780-1A9 cells to OTX-008 (Supplementary FIG. 1A). As shown in FIG. 3C, inhibition of protein degradation with the proteasome inhibitor bortezomib counteracts the inhibitory effects of OTX-008 on galectin-1 expression, suggesting that OTX-008 may facilitate galectin-1 degradation. A whole decrease galectin-1 expression was shown in the cytoplasm of SQ20B cells exposed to OTX-008 using immunofluorescence (FIG. 3D). Galectin-1 translocates from the cytoplasm to the nucleus upon exposure of OTX-008 (FIG. 3E). No change in GAL1 mRNA expression was observed in OTX-008 exposed cancer cells.

Effects of OTX-008 on the Extracellular Galectin-1/Semaphorin-3A System.

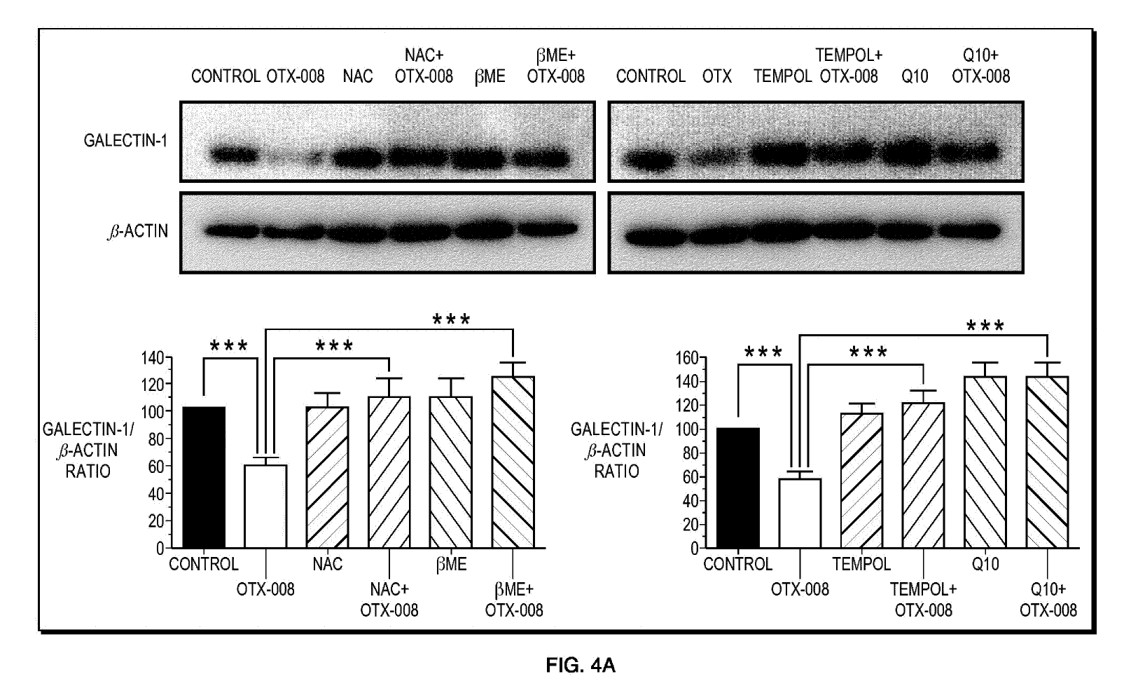

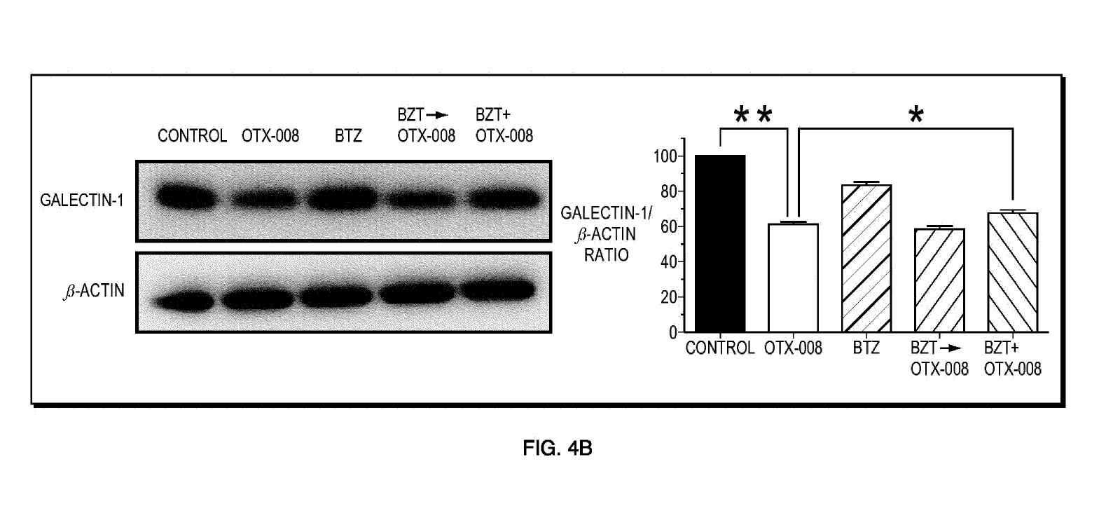

Galectin-1 may form extracellular lattices counteracting the inhibitory properties of semaphorin-3A on neuropilin-1, eventually leading to the modulation of the vascular endothelial growth factor (VEGFR) and platelet derived growth factor (PDGFR) receptors, fibronectin, and laminin activity SQ20B cells expressed significant mRNA and protein levels of semaphorin-3A and neuropilin-1, PDGFRa&(, VEGF A-D, and VEGFR1-3 (Supplementary FIG. 2A). Thus, SQ20B cells were selected to evaluate the effects of OTX-008 on galectin-1/semaphorin-3A interactions. No difference between SQ20B cells growing in plastic or fibronectin- and laminin-coated plates. Recombinant semaphorin-3A, galectin-1 and VEGF-A added to the media led to transient activations in p-AKT and p-ERK1/2, as well as p-S6 signaling suggesting that the galectin-1/semaphorin-3A system was functional in SQ20B cells (Supplementary FIG. 2B). Consistently, exogenous exposure to galectin-1 or semaphorin-3A resulted in different effects on cellular proliferation (FIG. 4A). Exogenous exposure of SQ20B cells to recombinant galectin-1 had not effects on SQ20B cell proliferation. However, increased concentration of lactose, a disaccharides sugar known to inhibit cellular interaction of galectin-1 with semaphorin-3A on neuropilin-1 led to significant inhibition of cellular proliferation. The effects of exogenous recombinant semaphorin-3A were tested, and showed a concentration-dependent inhibition of cellular proliferation. Interestingly, OTX-008 enhanced the antiproliferative effects of exogenous exposure to semaphorin-3A as well as lactose (FIG. 4A). As shown in FIG. 4B, exogenous exposures to recombinant galectin-1 and semaphorin-3A enhance invasion in SQ20B cells. Galectin-1 and semaphorin-3A induced invasion was inhibited by OTX-008 mimicking the effects of lactose in this model. Lactose prevents the inhibition of invasion induced by OTX-008 in SQ20B cells. UO126 that blocks the MAPK kinase pathway neither impact cellular invasion alone nor modify the anti-invasive effects of OTX-008. These data suggest that OTX-008 may at least in part interact with the extracellular galectin-1/semaphorin-3A system in cancer cells.

OTX-008 Induces ERK1/2-Dependent Cdc2 Inhibition.

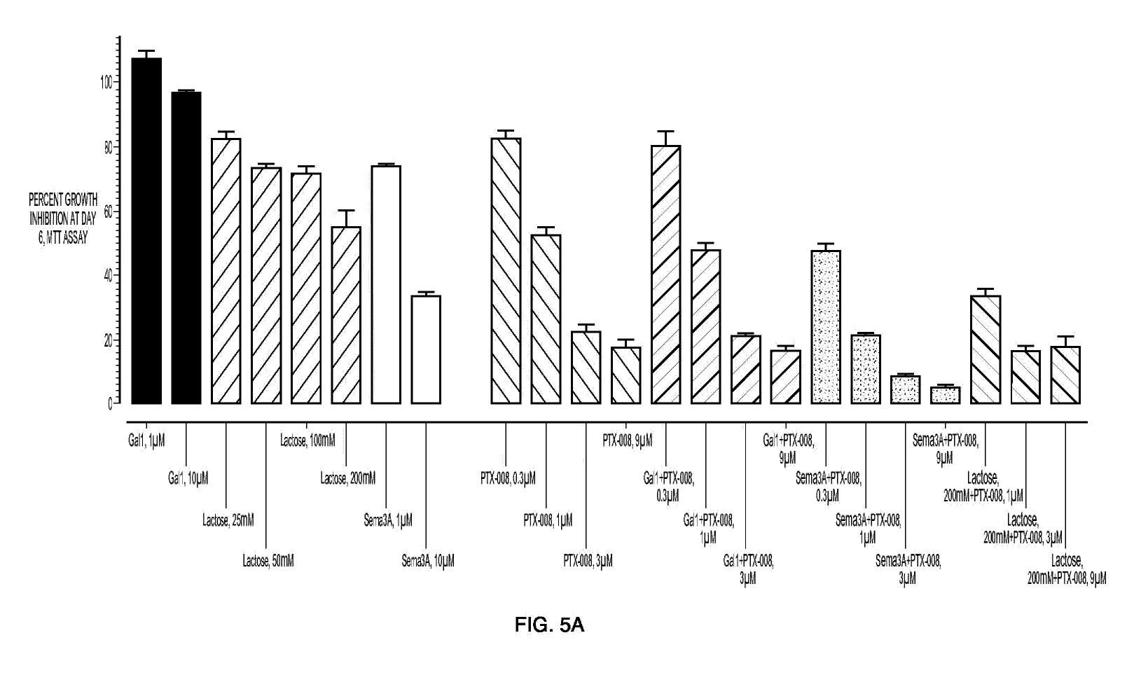

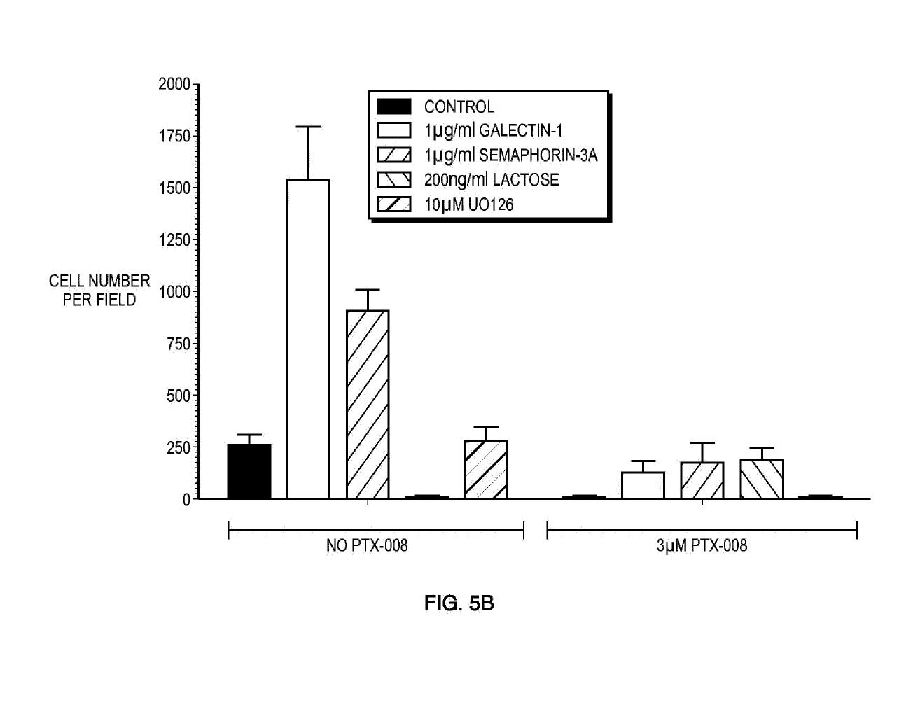

Intracytoplasmic galectin-1 was previously shown to stabilize intracellular GTP-Ras conformation, facilitating Ras downstream signaling. Signaling events following OTX-008 exposure in SQ20B cells displaying no EGFR, K-Ras, and B-Raf mutation were investigated (FIG. 5A). In SQ20B, exposure to OTX-008 progressively but sustainably inhibited p-ERK1/2 activity for at least 24 hours. OTX-008 also induced a transient activation of AKT^{s473} followed by a complete and sustained inhibition of p-AKT and downstream p-S6.

To study the mechanism of OTX-008-induced inhibition of cell proliferation, the effect of OTX-008 on cell cycle in SQ20B cells was examined (FIG. 5B). OTX-008 induced a dose-dependent accumulation of cells in G2/M phase of the cell cycle at concentrations associated with antiproliferative effects in SQ20B cells. Similar results were obtained in A2780-1A9 cell line (Supplementary FIG. 1B). OTX-008 had no direct effects on γH2AX expression suggesting no DNA lesions. Activation of cdc2 (cdk1) by de-phosphorylation of tyrosine 15 is required for G2/M transition. The kinase wee1 phosphorylates cdc2 on tyrosine 15 and becomes inactive when itself phosphorylated on several residues, such as serine 642. Thus, the level of phosphorylated cdc2 on tyrosine 15 and wee1 on serine 642 in SQ20B cell line were exposed to 3 μM OTX-008 for 72 h. As shown in FIG. 5C, exposure to OTX-008 in SQ20B cells inactivated cdc2 and was associated with decreased inactivating phosphorylation of wee1. Similar results were observed in A2780-1A9 (Supplementary FIG. 1C). Mild to no changes in chk1, chk2, and cdc25C phosphorylation levels were observed in OTX-008-treated SQ20B cells. These results suggested that OTX-008 activates wee1 leading to the phosphorylation of cdc2, which may in turn block entry in mitosis. In order to determine the potential relationship between the inhibition of MAPK signaling pathways and the effects on cell cycle by OTX-008, SQ20B cells were pretreated for 2 h with the ERK1/2 inhibitor UO126 followed by the OTX-008 addition for 72 hours. As shown in FIG. 5D, pretreatment with UO126 inhibited the increase of cdc2 phosphorylation mediated by OTX-008 exposure, suggesting that inhibiting MAPK pathways counteract the effect of OTX-008 on cdc2 phosphorylation. Taken together, these results showed that OTX-008 inhibition of mitosis entry in SQ20B cells is associated with a MAPK-dependent activation of wee1 and inhibition of cdc2.

OTX-008 Inhibits the Growth of Human Tumor Xenografts.

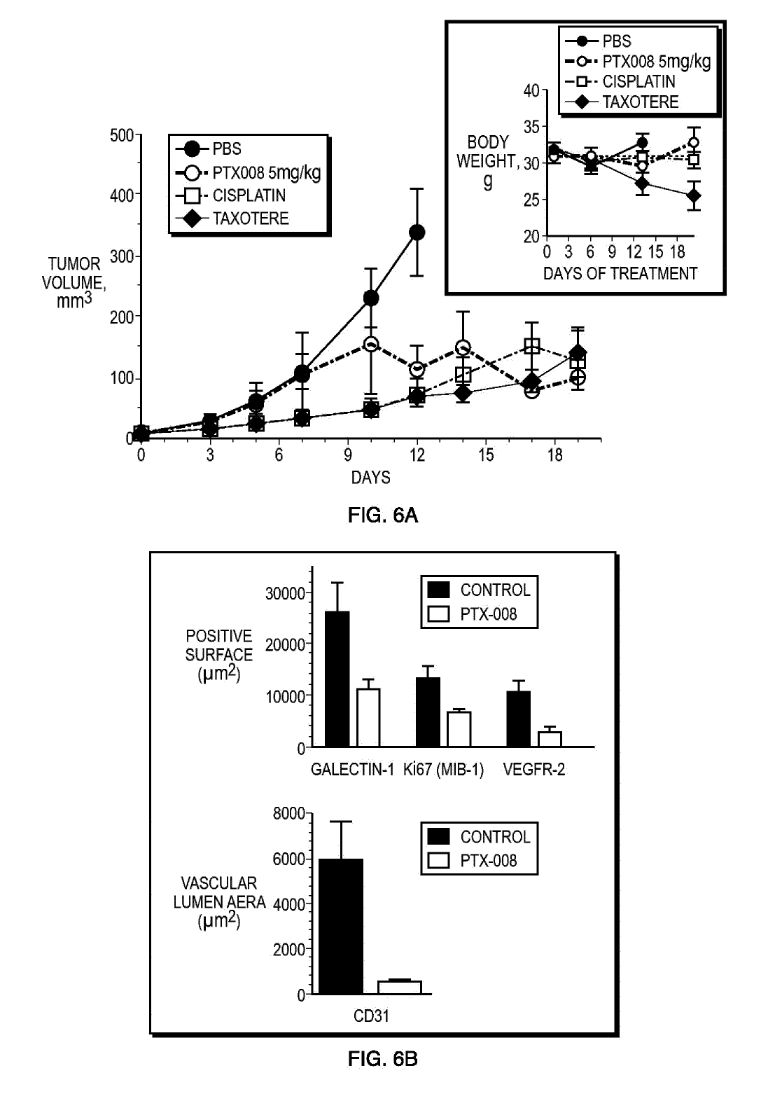

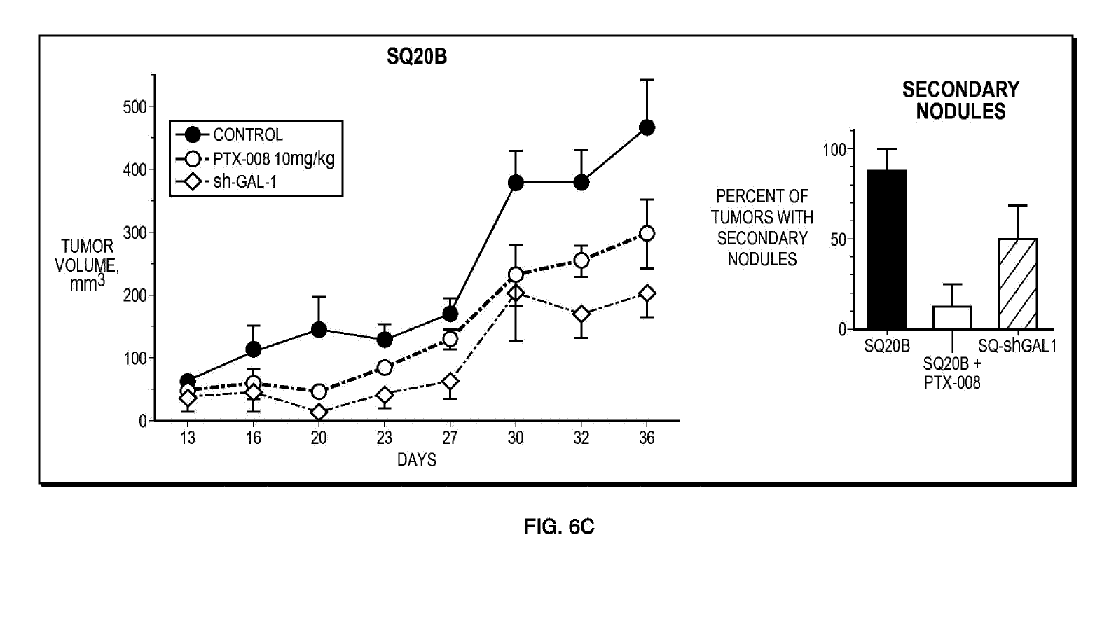

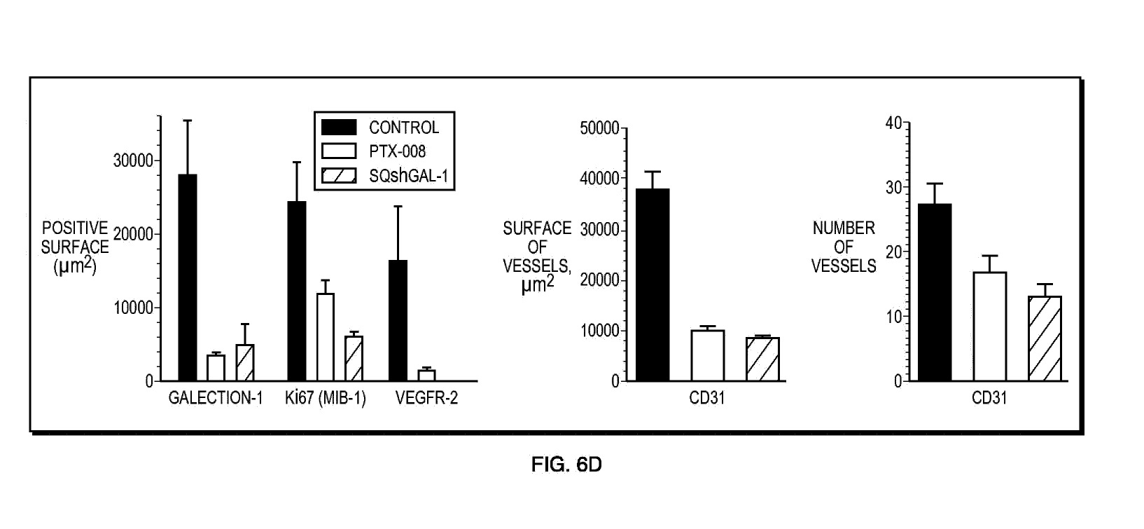

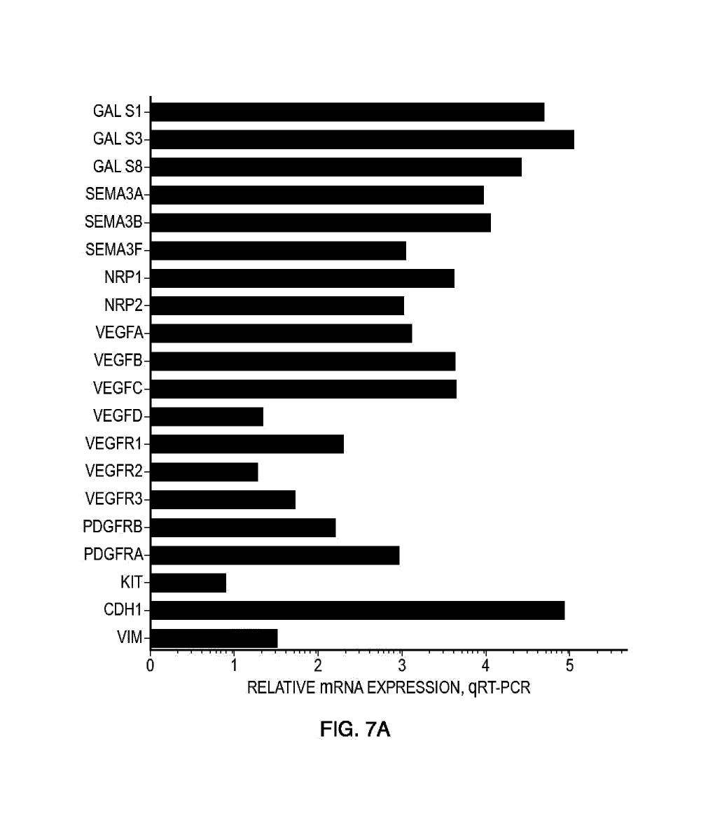

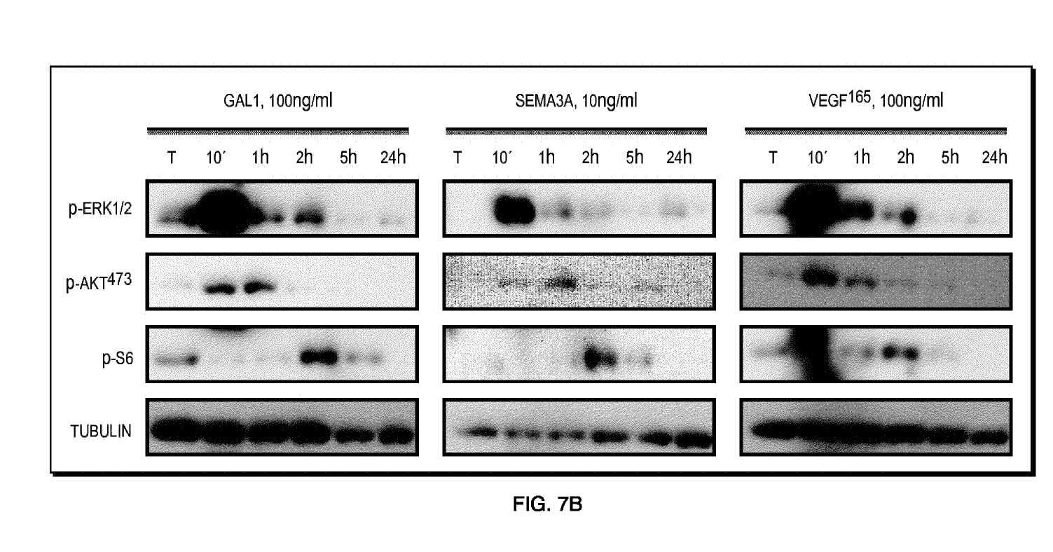

To investigate the effects of OTX-008 in xenografts, SQ20B and A2780-1A9 cancer cells were selected. Tumor growth inhibition was obtained using 5 mg/kg OTX-008 in A2780-1A9 ovarian xenografts. The antitumor effects of OTX-008 were comparable to that of cisplatin and docetaxel in A2780-1A9 tumors (FIG. 6A) but the antitumor effects of OTX-008 were observed at doses that had no toxicity in nude mice. Consistent with that observed in cultured cells, A2780-1A9 tumors treated with OTX-008 had significant lower galectin-1 expression in cancer cells as compared to non treated tumors (FIG. 6B). In A2780-1A9 tumors, proliferation as measured by Ki67 expression was significantly reduced (FIG. 6B). Also observed was a decreased number of micro-vessel in tumors associated with a decreased expression of VEGFR-2 in cancer cells. As vessel in treated tumors seems to be smaller than that in control, the vessels lumen surface area showing a significant decrease of the surface of vessels in OTX-008 treated mice as compared to control (FIG. 6B) were quantified.

As shown in FIG. 6C-right, OTX-008 given at a non-toxic dose also inhibited the growth of SQ20B xenografts. Interestingly, subcutaneous injections of SQ20B cells in mice resulted in the development of multiple subcutaneous metastases. In order to study the effect of OTX-008 treatment on metastasis, secondary tumor development was analyzed at mouse autopsies. As shown in FIG. 6C-left, OTX-008 significantly inhibited secondary tumor development in mice with only 14% of mice developing subcutaneous metastasis when treated with OTX-008 compared to >80% metastases in the control group. Consistent with data from A2780-1A9 tumors, treatment of SQ20B-xenograft bearing mice with OTX-008 resulted in a significant decrease galectin-1 expression and cell proliferation (Ki67) (FIG. 6D). In SQ20B tumors, the number and diameter of micro-vessels were also significantly reduced in OTX-008 treated tumors as compared to control (FIG. 6D).

Silencing Galectin-1 Recapitulates the Antitumor Effects of OTX-008.

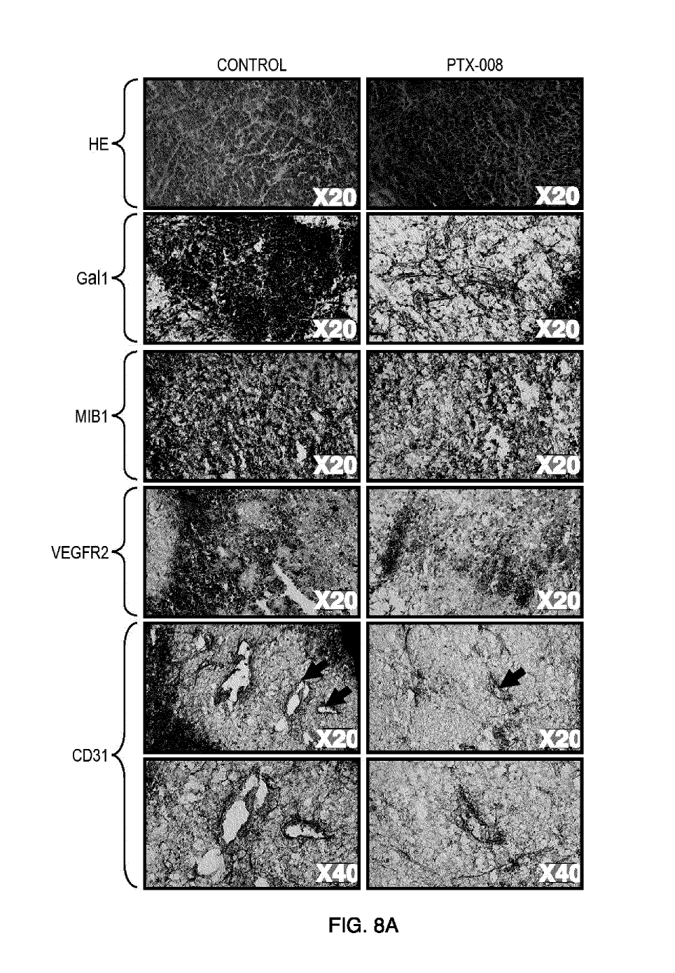

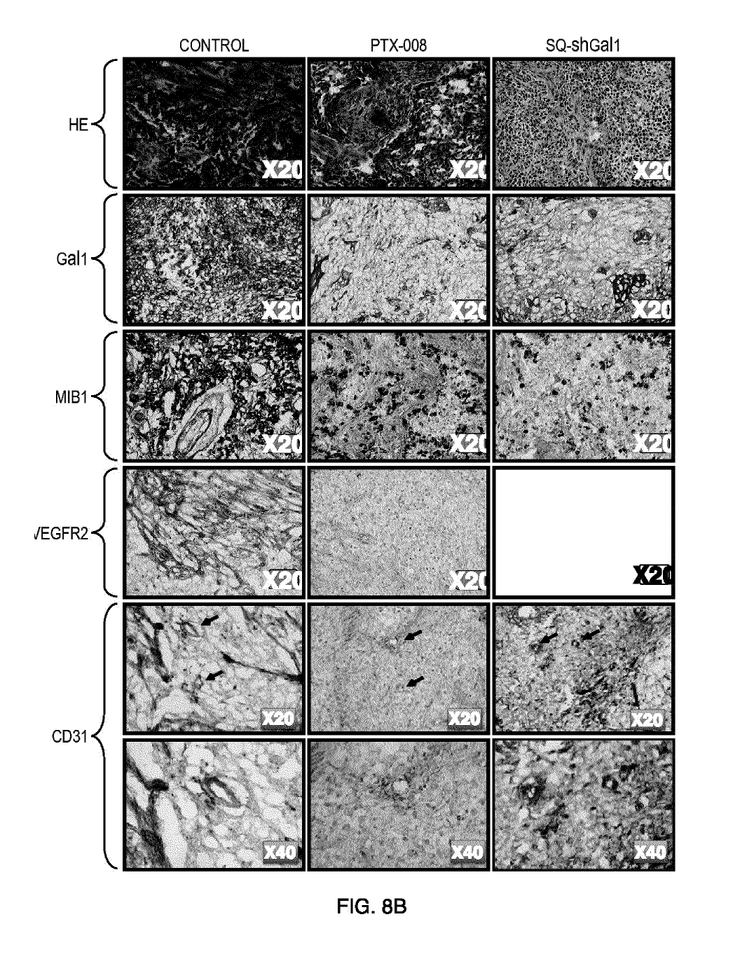

SQ-shGal1 cells were developed from SQ20B using a small hairpin GAL1-RNA silencing transfection. SQ-shGal1 cells display no difference in cell doubling-time and cloning capacity but expressed lower galectin-1 expression and invasive capacity in matrigel as compared to SQ20B (FIG. 8). Exposure of SQ-shGal1 cells to OTX-008 does not further increase its antiproliferative effects in vitro. SQ-shGal1 cells were subcutaneously injected in nude mice and the growth of SQ-shGal1 tumors was significantly lower than that of SQ20B tumors (FIG. 6C-right). As shown in FIG. 6C-left, silencing galectin-1 expression in mice was associated with a decreased development of secondary tumors as compared to parental SQ20B tumors. SQ-shGal1 cancer cells in tumors expressed lower level of Ki67 and VEGFR-2 expressions (FIG. 6D). As previously described with OTX-008, SQ-shGal1 tumors had significantly lower number and small micro-vessels as compared to SQ20B xenografts (FIG. 6D). Those data showed that selective inhibition of galectin-1 using shGal-1 RNA yields similar feature than that observed in OTX-008 treated tumors.

As shown herein, OTX-008, a synthetic-peptidomimetic designed to bind the amphipathic β-sheet conformation of galectin-1, inhibited proliferation in a wide range of cancer cells and delayed tumor growth in A2780-1A9 human ovarian cancer and SQ20B human squamous cell head-and-neck cancer xenograft models. In vitro, OTX-008 treatment resulted in decreased galectin-1 protein expression and translocation of galectin-1 from the cytoplasm to the nucleus. In vivo, immunohistochemical analysis of OTX-008 treated tumors showed decreased galectin-1 expression and decreased Ki67 expression, consistent with the antiproliferative effects observed with OTX-008 in cultured cells, as well as normalization of tumor vasculature. Results from xenograft models were consistent with previous reports of OTX008 treatment of MA148 human ovarian cancer xenografts and the B16F10 murine melanoma model.

SQ20B cells, which were the most sensitive to OTX-008 among the cell lines tested, were used to explore the cellular effects of OTX-008 treatment. It is shown herein that OTX-008 treatment led to a progressive inhibition of ERK1/2 signaling. Previous reports showed that intracellular galectin-1 might favor the H-Ras- and K-Ras-GTP conformations that modulate the signal output of the mitogen activated kinase survival pathway in cancer cells. Inhibition of ERK1/2-, AKT-, and S6-phosphorylation as a result of down expression of galectin-1 was also reported by Thijssen et al. in endothelial cells using galectin-1 knockdown constructs and anginex, a galectin-1 binding peptide. OTX-008 exposure was observed to induce nuclear translocation of galectin-1. Previous studies demonstrated that galectin-1 could co-immunoprecipitate in the nucleus with Gemin4, a member of the survival of motor neuron protein complex that plays a role in the biogenesis of microRNA ribonucleoprotein. Nuclear extracts depleted of galectin-1 were also shown to lose splicing capacity, suggesting the important role of galectin-1 in the splicing pathway and spliceosome assembly. As OTX-008 may modulate galectin-1 in the nucleus, further studies may be required to investigate potential effects on microRNA ribonucleoprotein biogenesis.

It is shown herein that concentrations of OTX-008 required to inhibit proliferation were proportional to galectin-1 expression in cancer cells. Several studies have shown that galectin-1 could play an important role in invasion and migration, which are also increased in cells with epithelial-to-mesenchymal transition. It is shown herein that cancer cells with mesenchymal phenotype display higher galectin-1 expression than epithelial cells. Consistently, OTX-008 displayed antiproliferative effects at low concentrations in cancer cells with an epithelial phenotype, but required higher concentrations to inhibit the growth of cancer cells displaying a mesenchymal phenotype. Cellular effects of OTX-008 in epithelial cancer cells appear to be related at least in part to its interaction with the galectin-1/semaphorin-3A/neuropilin system. In SQ20B cells that display a functional galectin-1/semaphorin-3A/neuropilin system, exposure to exogenous galectin-1 slightly increased cellular proliferation but significantly enhanced cellular invasion. Inhibiting galectin-1 expression using galectin-1 (LGALS1) shRNA in this model showed no effect on the cellular doubling time but significantly reduced the invasive capacity of SQ20B cells. Consistently, a lower number of subcutaneous metastasis was observed in SQ20B-shGAL1 xenografts as compared to its non-silenced counterpart. Altogether, these data suggest that galectin-1 might modulate cellular proliferation and invasion in this model. In SQ20B cells, OTX-008 inhibits proliferation and invasion. While no change in the antiproliferative effects of OTX-008 were observed in the presence of exogenous galectin-1, the enhanced invasiveness induced by galectin-1 was fully inhibited by OTX-008 in SQ20B cells. Consistently, OTX-008 also reduced the number of subcutaneous metastasis in SQ20B xenografts. To better understand the effects of OTX-008 on galectin-1 functions, lactose was used. Lactose was previously shown to prevent neuropilin-1-galectin-1 interactions. In SQ20B cells, OTX-008 enhanced the effects of lactose on cellular proliferation and counteracted the inhibitory effects of lactose on invasion. As shown herein, exogenous semaphorin-3A inhibits cancer cell proliferation and enhances invasion. OTX-008 potentiates the antiproliferative effects of semaphorin-3A and inhibits the semaphorin-3A-induced invasion. Altogether, these data suggest that OTX-008 can interact with the galectin-1/semaphorin-3A/neuropilin system, counteracting its pro-proliferative and invasive potential in cancer cells. Mechanisms by which OTX-008 interacts with extracellular galectin-1 remain unclear. In addition to direct binding to galectin-1, OTX-008 may also oxidize the extracellular galectin-1 inhibiting the dimer formation and further activation of neuropilin-1.

As shown herein, OTX-008 treatment reduced galectin-1 expression and delayed tumor-growth in A2780-1A9 and SQ20B xenograft models. The reduced Ki67 expression in cancer cells was consistent with the antiproliferative effects observed with OTX-008 in cultured cells. Interestingly, the decrease galectin-1 expression in xenografts was associated with a significant reduction of tumor angiogenesis. Anginex, a galectin-1 binding peptide that served for the design of OTX-008, was previously shown to inhibit tumor angiogenesis. Recent data also demonstrated that galectin-1 knockdown expression in cancer cells could reduce tumor angiogenesis by inhibiting endothelial cell migration and proliferation in xenograft models. As shown herein, immunostaining showed that the sizes of microvessels and the VEGFR2 expression in endothelial cells were significantly reduced in xenograft models treated with OTX-008. Galectin-1 down-expression using shRNA recapitulated most of the effects of OTX-008 on tumor angiogenesis. These data strongly suggest that OTX-008 may interfere with the galectin-1/VEGFR expression in endothelial cells resulting in normalization of microvessels.

In summary, the data herein shows that OTX-008, a galectin-1-targeted compound, reduces galectin-1 expression, inhibits cancer cell proliferation, prevents invasion, and inhibits angiogenesis. The results disclosed herein suggest that galectin-1 is a target for anticancer therapy, provide insights on the mechanism of action of OTX-008, and support further development of this drug as an anticancer agent.

1. A method of inhibiting a galectin-1-associated pathway in a mammalian cell, the method comprising contacting a mammalian cell with an effective amount of a galectin-1-targeting compound.

2. The method of claim 1, wherein the method is a method of treating cancer in a subject having cancer, comprising contacting one or more cancerous cells in the subject with a therapeutically effective amount of a galectin-1 targeting compound.

3. The method of claim 1, wherein the method is a method of inhibiting at least one of growth and proliferation of a cancer cell, comprising contacting said cancer cell with a therapeutically effective amount of a galectin-1 targeting compound.

4. The method of claim 1, wherein the method is a method of inhibiting at least one of tumor growth and metastasis in a patient having a tumor, comprising contacting said tumor with a therapeutically effective amount of a galectin-1 targeting compound.

5. The method of claim 1, wherein the method is a method of inhibiting at least one of galectin-1 activity and expression, comprising contacting a cell with a therapeutically effective amount of a galectin-1 targeting compound.

6. The method of claim 1, wherein the galectin-1 targeting compound is OTX-008:

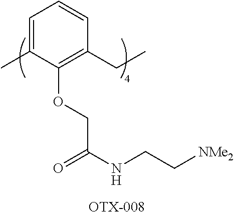

7. A method of inhibiting a galectin-1-associated pathway in a mammalian cell, the method comprising contacting a mammalian cell with an effective amount of OTX-008.

8. The method of claim 7, wherein the method is a method of treating cancer in a subject having cancer, comprising contacting one or more cancerous cells in the subject with a therapeutically effective amount of OTX-008.

9. The method of claim 7, wherein the method is a method of inhibiting at least one of growth and proliferation of a cancer cell comprising contacting said cancer cell with a therapeutically effective amount of OTX-008.

10. The method of claim 7, wherein the method is a method of inhibiting at least one of tumor growth and metastasis in a patient having a tumor, comprising contacting said tumor with a therapeutically effective amount of OTX-008.

11. The method of claim 7, wherein the method is a method of inhibiting at least one of galectin-1 activity and expression, comprising contacting a cell with a therapeutically effective amount of OTX-008.

12. The method of claim 2, wherein the treatment of the cancer includes at least one of preventing or slowing the growth of the cancer, preventing the spread of a tumor associated with the cancer, preventing the spread of one or more metastases associated with the cancer, reducing the size of a tumor associated with the cancer, and preventing the recurrence of cancer treated previously.

13. The method of claim 8, wherein the therapeutically effective amount of OTX-008 comprises a pharmaceutical composition.

14. The method of claim 13, wherein the pharmaceutical composition is administered by at least one route selected from the group consisting of intravenously, subcutaneously, intradermally, parenterally, and intramuscularly.

15. The method of claim 13, wherein the pharmaceutical composition is administered in combination with at least one other mode of therapy selected from the group consisting of radiotherapy, chemotherapy, and surgery.

16. (canceled)

17. The method of claim 8, wherein the cancer is selected from the group consisting of ovarian cancer, squamous cell carcinoma, a cancer of the digestive system, stomach cancer, liver cancer, colon cancer, a cancer of the thyroid, a cancer of the endometrium, adenocarcinoma of the endometrium, uterine cancer, uterine adenocarcinomac a uterine smooth muscle tumor, breast cancer, prostate cancer, bladder cancer, a head cancer, a neck cancer, a glioma, a kidney cancer, pancreatic cancer, pancreatic ductal adenocarcinoma, nonsmall-cell lung cancer, and melanoma.

18. A method of inhibiting the galectin-1/semaphorin-3A system in a cancer cell, the method comprising contacting the cell with an effective amount of a galectin-1-targeting compound.

19. (canceled)

20. The method of claim 1, wherein the galectin-1-targeting compound is administered in a dose selected from the group consisting of about 1 microgram to about 1,000 micrograms, about 25 micrograms to about 500 micrograms, about 50 to about 250 micrograms, about 2 micrograms to about 100 micrograms, about 5 micrograms to about 75 micrograms, about 10 micrograms to about 50 micrograms, and about 20 micrograms to about 40 micrograms.

21. A method of treating a galectin-1-overexpressing cancer in a patient having a galectin-1-overexpressing cancer, the method comprising administering to the patient a therapeutically effective amount of a composition comprising at least one galectin-1-targeting compound.

22. The method of claim 22, wherein the galectin-1-targeting compound is OTX-008.
